(19) United States
(12) Patent Application Publication
(10) Pub. No.: US 2012/0172246 A1
(43) Pub. Date: Jul. 5, 2012

(54) Detection of Nucleic Acids

(75) Inventors: Quan N. Nguyen, San Ramon, CA (US); Robert J. Lipshutz, Palo Alto, CA (US); Gary K. McMaster, Ann Arbor, MI (US); Yunqing Ma, San Jose, CA (US); Yen-Chieh Wu, Zhubei (TW); Audrey Lin, San Jose, CA (US); Chunfai Lai, Fremont, CA (US); Cung-Tuong Nguyen, Milpitas, CA (US)

(73) Assignee: AFFYMETRIX, INC., Santa Clara, CA (US)

(21) Appl. No.: 13/342,392

(22) Filed: Jan. 3, 2012

Related U.S. Application Data
(60) Provisional application No. 61/429,054, filed on Dec. 31, 2010.

(51) Int. Cl. C40B 30/04; C12Q 1/68

(52) U.S. Cl. 506/9; 435/6.11

(57) ABSTRACT

Methods of detecting various types of nucleic acids, including methods of detecting two or more nucleic acids in multiplex branched-chain DNA assays, are provided. Detection assays may be conducted at least in vitro, in cellulo, and in situ. Nucleic acids which are optionally captured on a solid support are detected, for example, through cooperative hybridization events that result in specific association of a label probe system with the nucleic acids. Various label probe system embodiments are provided. Compositions, kits, and systems related to the methods are also described.

### PRIORITY CLAIM

This application claims priority to U.S. Provisional Patent Application No. 61/429,054 filed Dec. 31, 2010, the subject matter of which is being incorporated herein by reference in its entirety.

### FIELD OF THE INVENTION

Disclosed are methods, compositions and kits for detection of nucleic acids, including methods for detecting the presence of two or more nucleic acids simultaneously in a single sample. Detection may be, for instance, in vivo, in cellulo or in situ. Detection may include or be directed towards detection and quantitation of single nucleotide polymorphisms, i.e. SNP detection, copy number, micro-RNA, siRNA, transcription level determination, and other similar genetic information.

### BACKGROUND OF THE INVENTION

A variety of techniques for detection of nucleic acids involve a first step of capturing or binding of the target nucleic acid or nucleic acids to a surface through hybridization of each nucleic acid to an oligonucleotide (or other nucleic acid) that is attached to the surface. For example, DNA microarray technology, which is widely used to analyze gene expression, copy number determination and single nucleotide polymorphism detection, relies on hybridization of DNA targets to preformed arrays of polynucleotides. (See, e.g., Lockhart and Winzeler, “Genomics, gene expression and DNA arrays,” Nature, 405:827-36 (2000); Gerhold et al. “Monitoring expression of genes involved in drug metabolism and toxicology using DNA microarrays,” Physiol. Genomics, 5:161-70, (2001); Thomas et al. “Identification of toxicologically predictive gene sets using cDNA microarrays,” Mol. Pharmacol., 60:1189-94 (2001); and Epstein and Butow, “Microarray technology—enhanced versatility, persistent challenge,” Curr. Opin. Biotechnol., 11:36-41 (2000)). Single nucleotide polymorphism (SNP) has been used extensively for genetic analysis. Fast and reliable hybridization-based SNP assays have been developed. (See, Wang et al., Science, 280:1077-1082, 1998; Gingeras, et al., Genome Research, 8:435-448, 1998; and Halushka, et al., Nature Genetics, 22:239-247, 1999; incorporated herein by reference in their entireties). Methods and arrays for simultaneous genotyping of more than 10,000 SNPs, and more than 100,000 SNPs, have been described, for example, in Kennedy et al., Nat. Biotech., 21:1233-1237, 2003, Matsuzaki et al., Genome Res., 14(3):414-425, 2004, and Matsuzaki et al., Nature Methods, 1:109-111, 2004 (all of which are incorporated herein by reference in their entireties for all purposes).

As provided by the Human Genome Project Information website ((genomics).energy.gov), “Although more than 99% of human DNA sequences are the same, variations in DNA sequence can have a major impact on how humans respond to disease; environmental factors such as bacteria, viruses, toxins, and chemicals; and drugs and other therapies. This makes SNPs valuable for biomedical research and for developing pharmaceutical products or medical diagnostics. SNPs are also evolutionarily stable—not changing much from generation to generation—making them easier to follow in population studies.” However, it is pointed out that SNPs do not cause disease and they are not absolute indicators of disease development. However, based on SNP identity and linkage analysis studies performed which link the occurrence of various SNPs in a person's genome to diseases found in various sub-populations, SNPs can help determine the likelihood that a person might develop various diseases or illnesses. Studies using panels of human SNPs to identify evidence for linkage between genomic regions and disease phenotypes have been described. (See, for example, Boyles et al., Am. J. Med. Genet. A., 140(24):2776-85 (2006), Klein et al., Science, 308: 385 (2005), Papassotiropoulos et al., Science, 314:475-478 (2006), Craig and Stephan, Expert Rev. Mol. Diagn., 5(2):159-70 (2005) and Puffenberger et al., Proc. Nat'l. Acad. Sci. USA, 101:11689-94 (2004)).

One type of genetic characteristic or information which can now be tested for is the genetic copy number state of particular regions or segments of the chromosome. Copy Number State (CNState) values are typically determined in series for each chromosome within the genome of the experimental genetic sample to find segments where chromosomal material has incurred a gain or loss of copies of genetic material. Genetic testing also enables detection of area of loss of heterozygosity (LOH) or LCSH, and/or may reveal of areas of non-interger CNState, e.g. copy number mosaicism. Long Contiguous Stretches of Homozygosity (LCSH) in a genomic region (stretch) indicates a region in which the Copy Number is neutral (two copies) but which displays a Loss of normal heterozygosity, and thus is homozygous for the measured SNP allele information. These are areas where a mixture of samples provide data relating to a specific chromosomal region which varies in integer copy number between the two admixed samples. This is also known as “mosaicism,” e.g. a genetic phenomenon wherein the determined copy number of the genetic marker is not a near a whole integer, but rather is between two whole integers. These Copy Number Variations (CNVs) are typically discovered using techniques such as fluorescent in situ hybridization (FISH), comparative genomic hybridization (CGH) or virtual karyotyping using oligonucleotide microarrays. Some CNVs have been associated with various diseases and this is an on-going and exciting field of research. For instance, elevated levels of EGFR gene has been found in non-small cell lung cancer, a higher level of the gene CCL3L1 has been associated with a lower susceptibility to human HIV, and various other CNVs have been associated with diseases such as autism, schizophrenia and idiopathic learning disability. In particular, a major focus in cytogenetics research is on Uniparental Disomy (UPD) events where a child inherits two copies of chromosomal material from one parent and nothing from the other. These UPD events are known to be linked with recessive disorders and also cause developmental disorders due to gene imprinting. These events occur without associated copy number changes. For instance approximately 30% of Prader-Willi cases are associated with paternal UPD of chromosome 15q, 2-3% of Angelman Syndrome are associated with maternal UPD of 15q, 10-30% of Beckwith-Wiedemann Syndrome are associated with maternal UPD of 11p15, and 5% of Silver-Russell Syndrome are associated with maternal UPD of chromosome 7.

A typical DNA microarray contains a large number of spots or features, with each spot or feature containing oligonucleotides which have a single oligonucleotide sequence, each intended to be complementary to and to hybridize to a specific nucleic acid target. For example, the GeneChip® microarray available from Affymetrix (Santa Clara, Calif.) can includes millions of features, with each feature containing multiple copies of a different single 25mer oligonucleotide sequence. (See, Lockhart et al., “Expression monitoring by hybridization to high-density oligonucleotide arrays,” Nature Biotechnology, 1996, 14(13):1675-80; Golub et al., “Molecular classification of cancer: class discovery and class prediction by gene expression monitoring,” Science, 1999, 286(5439), 531-7, each of which is incorporated herein by reference in their entirety for all purposes).

In another approach, longer oligonucleotides are used to form the spots in the microarray. For example, instead of short oligonucleotides, longer oligonucleotides or cDNAs can be used to capture the target nucleic acids. Use of longer probes can provide increased specificity, but it can also make discrimination of closely related sequences difficult. Adjusting the length of the oligonucleotide probe to provide the desired specificity and sensitivity often proves extremely difficult. This further requires precise adjustment of hybridization temperature and other solution-phase parameters. When attempting to detect multiple targets simultaneously in one assay, or for instance one microarray, all of these variables must be considered and optimized to increase the robustness of the assay and the yield of assured genotyping calls.

Many different avenues of research have been investigated to address these issues of specificity and sensitivity of such hybridization-based genetic assays. For instance, the use of oligonucleotide analogs have been investigated which increase the melting temperature at which the target hybridizes to the capture oligonucleotide.

Improved methods for hybridizing oligonucleotide probes in a specific manner with high affinity and desired sensitivity to target nucleic acids are thus desirable. Among other aspects, presently disclosed are methods that address these limitations and which permit rapid, simple, and highly specific capture of multiple nucleic acid targets simultaneously.

Global gene expression profiling and other technologies have identified a large number of genes whose expression is altered in diseased tissues or in tissues and cells treated with pharmaceutical agents. (See, Lockhart and Winzeler, (2000) “Genomics, gene expression and DNA arrays,” Nature, 405:827-36, and Gunther et al., (2003) “Prediction of clinical drug efficacy by classification of drug-induced genomic expression profiles in vitro,” Proc. Natl. Acad. Sci. USA, 100:9608-13). The capability of measuring the expression level of all of the expressed genes in a cell enables linking of these expression patterns to specific diseases. Therefore, gene expression is increasingly being used as a biomarker or prognosticator of disease, determination of the stage of disease, and indicator of prognosis. (See, Golub et al., (1999) “Molecular classification of cancer: class discovery and class prediction by gene expression monitoring,” Science, 286:531-7). Other applications of gene expression analysis and detection include, but are not limited to, target identification, validation and pathway analysis (Roberts et al. (2000) “Signaling and circuitry of multiple MAPK pathways revealed by a matrix of global gene expression profiles,” Science, 287:873-80), drug screening (Hamadeh et al., (2002) “Prediction of compound signature using high density gene expression profiling,” Toxicol. Sci., 67:232-40), and studies of drug efficacy, structure-activity relationship, toxicity, and drug-target interactions (Gerhold et al., (2001) “Monitoring expression of genes involved in drug metabolism and toxicology using DNA microarrays,” Physiol. Genomics, 5:161-70 and Thomas et al., (2001) “Identification of toxicologically predictive gene sets using cDNA microarrays,” Mol. Pharmacol., 60:1189-94). As biomarkers are identified, their involvement in disease management and drug development will need to be evaluated in higher throughput and broader populations of samples. Simpler and more flexible expression profiling technology that allows the expression analysis of multiple genes with higher data quality and higher throughput is therefore needed.

Levels of RNA expression have traditionally been measured using Northern blot and nuclease protection assays. However, these approaches are time-consuming and have limited sensitivity, and the data generated are more qualitative than quantitative in nature. Greater sensitivity and quantification are possible with reverse transcription polymerase chain reaction (RT-PCR) based methods, such as quantitative real-time RT-PCR, but these approaches have low multiplex capabilities. (See, Bustin, (2002) “Quantification of mRNA using real-time reverse transcription PCR (RT-PCR): trends and problems,” J. Mol. Endocrinol., 29:23-39, and Bustin and Nolan, (2004) “Pitfalls of quantitative real-time reverse-transcription polymerase chain reaction,” J. Biomol. Tech., 15:155-66). Microarray technology has been widely used in discovery research, but its moderate sensitivity and its relatively long experimental procedure have limited its use in high throughput expression profiling applications (Epstein and Butow, (2000) “Microarray technology—enhanced versatility, persistent challenge,” Curr. Opin. Biotechnol., 11:36-41).

Most of the current methods of mRNA quantification require RNA isolation, reverse transcription, and target amplification. Each of these steps has the potential of introducing variability in yield and quality that often leads to low overall assay precision. Recently, a multiplex screening assay for mRNA quantification combining nuclease protection with luminescent array detection was reported. (See, Martel et al., (2002) “Multiplexed screening assay for mRNA combining nuclease protection with luminescent array detection,” Assay Drug Dev. Technol., 1:61-71). Although this assay has the advantage of measuring mRNA transcripts directly from cell lysates, limited assay sensitivity and reproducibility were reported. Another multiplex mRNA assay without the need for RNA isolation was also reported in Tian et al., entitled “Multiplex mRNA assay using electrophoretic tags for high-throughput gene expression analysis.” (Nucleic Acids Res., 32:126, 2004). This assay couples the primary INVADER® mRNA assay with small fluorescent molecule Tags that can be distinguished by capillary electrophoresis through distinct charge-to-mass ratios of Tags. However, this assay requires the use of a specially designed and synthesized set of eTagged signal probes, complicated capillary electrophoresis equipment, and a special data analysis package.

Another relatively new and exciting area of genetic research involves the study of micro-RNA, or miRNA. MicroRNA are short ribonucleic acid molecules which have been shown to be post-transcriptional regulators that bind complementary oligonucleotide sequences of mRNA transcripts. Upon binding, typically at the 3 Untranslated Regions (UTR), gene silencing may occur. Typically miRNA are about 15-25 nucleotides in length and a single miRNA could silence hundreds of mRNA transcripts inside the cell. Since the turn of the century, it has been recognized the miRNA play a vital role in gene regulation, through mechanisms including transcript degradation, sequestering of transcripts, and translational suppression or even up-regulation. Thus, aberrant expression of miRNA can lead to various disease states. Various scientific reports have been surfacing which in fact link aberrant miRNA expression to disease. This has led to the realization that certain diseases, such as cancer, heart disease and central nervous system abnormalities, may be addressed by controlling miRNA levels in the cell. (See, for instance, Heneghan et al., “MicroRNAs as Novel Biomarkers for Breast Cancer,” J. Oncology, 2010, ID 950201, Ryan et al., “Genetic variation in micrRNA networks: the implications for cancer research,” Nat. Rev. Cancer, 10(6):389-402 (2010), O'Connell et al., “Physiological and pathological roles for microRNAs in the immune system,” Nat. Rev. Immunol., 10:111-122, (2010), and Inui et al., “MicroRNA control of signal transduction,” Nat. Rev. Mol. Cell Biol., 11:252-263 (2010), all of which are incorporated herein by reference in their entirety for all purposes).

Another common technique for detecting genetic abnormalities is Fluorescence In Situ Hybridization (FISH). The FISH technique can be used to detect genetic abnormalities in nearly any type of tissue. In FISH analyses, single-stranded nucleic acids which are fluorescently labeled are allowed to bind to specific regions of the chromosome, and then examined through a microscope. Chromosome samples must first be fixed onto a slide, the labeled probe then hybridized to the chromosomes and then visualization is achieved through various enzyme-linked label-based detection protocols. Generally, the resolution of FISH analysis is on the order of detection of 60 base pairs (bp) up to 100 kilobase pairs of DNA. In contrast, CGH can detect abnormalities on the scale of 100 bp. Thus, the FISH analysis, though the golden standard today in cytogenetics, is time consuming and requires many steps, and in the end only provides 60 bp to 100 kb resolution.

Another genetic analysis product, called QUANTIGENE® (Panomics, Fremont, Calif.), is able to specifically bind and detect dozens of target sequences in a single sample. See, for instance, U.S. patent application Ser. Nos. 11/433,081 (allowed), 11/431,092, 11/471,025 (allowed), all of which are incorporated herein by reference in their entirety for all purposes. General protocols and user's guides on how the QUANTIGENE® system works and explanation of kits and components may be found at the Panomics website (see, www.panomics.com/index.php?id=product_{—}1#product_lit_{—}1). Specifically, user's manual, “QUANTIGENE® 2.0 Reagent System User Manual,” (2007) provided at the Panomics website is incorporated herein by reference in its entirety for all purposes (see, panomics.com/downloads/UM13074_QG2Manual_RevB_{—}080102. pdf and other addendums and FFPE Method Proficiency Kit User Manual and addendums also available from the Panomics website, for instance as the User Manual for FFPE available here: panomics.com/downloads/UM13898_QGFFPEProfManual_RevC_{—}071209.pdf, all of which are specifically incorporated herein by reference in their entirety for all purposes).

The QUANTIGENE® technology allows unparalleled signal amplification capabilities that provide an extremely sensitive assay. For instance, it is commonly claimed that the limit of detection in situ for mRNA species is about 20 copies of message per cell. However, in practice the limit of detection, due to the variability in the assay, is generally found to be around 50-60 copies of message per cell. This limit of detection limits the field of research since 80% of mRNAs are present at fewer than 5 copies per cell and 95% of mRNAs are present in cells at fewer than 50 copies per cell. As mentioned above, to arrive at this sensitivity, other approaches are very time consuming and complicated. Other technologies rely on the use of a panel of various enzymes and are affected by the fixation process of FFPE. In contrast, the QUANTIGENE® technology, such as QUANTIGENE® 2.0 and ViewRNA, is very simple, efficient and is capable of applying up to 400 labels per 50 base pairs of target. This breakthrough technology allows efficient and simple detection on the level of even a single mRNA copy per cell. Coupling this technology to detection of both mRNA and protein species will propel this field of research into heretofor inaccessible areas of study.

Among other aspects, the present invention provides methods that overcome the above noted limitations and permit rapid, simple, and sensitive detection of multiple mRNAs (and/or other nucleic acids) simultaneously. The present disclosure is related to U.S. Provisional Patent Application No. 61/360,887 which was filed on Jul. 1, 2010 and is expressly incorporated herein by reference in its entirety for all purposes. Other related applications include U.S. Provisional Patent Application Nos. 61/361,007 and 61/360,912, all of which are expressly incorporated herein by reference in their entirety for all purposes. A complete understanding of the invention will be obtained upon review of the following.

### SUMMARY OF THE INVENTION

Methods of detecting a target nucleic acid sequence are presented, including associated compositions, kits and systems. In general, methods including incubating a sample comprising at least one target nucleic acid with at least two label extender probes each comprising a different L-1 sequence and a label probe system with the sample comprising or suspected of comprising the target nucleic acid sequence, wherein at least one L-1 sequence is complementary to the at least one strand of the target nucleic acid sequence. The at least one L-1 sequence may comprise one or more locked nucleic acids which may be constrained ethyl nucleic acid(s) (cEt). In such embodiments, label extender pairs may be designed to bind to both sense and anti-sense strands of a double-stranded DNA or RNA target. The sample may be, for instance, purified chromosomes and the target may be double stranded DNA. The sample may comprise multiple target nucleic acid sequences, and each assay then comprising a different set of label extender probes designed to hybridize differentially to each target nucleic acid sequence. Therefore, label extender probes may comprise an allele-specific label probe and a non-allele-specific probe, wherein the non-allele-specific probe is designed to hybridize less stringently to the complimentary strand of the target sequence. This approach is used to increase the specificity and stringency of the assay. Further, the sample may be pre-incubated with amplifier and/or pre-amplifier probes to “adsorb” any non-specific hybridization interactions prior to conducting the assay. Finally, an additional set of label extender probes may be designed to hybridize directly upstream or downstream of the target nucleic acid as a control.

Targets useful in the present assay systems may be one or more of double-stranded DNA, miRNA, siRNA, mRNA, and single-stranded DNA. The disclosed methods may be performed in situ, in cellulo, in vitro, or in any number of different contexts due to the very flexible nature of the components. In fact, the sample may purified chromosomes, as typically analyzed in traditional karyotyping assays. Alternatively, the sample may be cells obtained from a cell culture or tissue culture medium.

The assays, embodiments and systems disclosed may be easily altered for multiplex reactions, i.e. wherein multiple targets are detected in a single sample. The sample may comprise a target nucleic acid comprising at least two single nucleotide polymorphisms, or multiple target nucleic acids. Due to the inherent flexibility of the present assays, methods, embodiments, compositions and systems, it is shown that the label extenders may be designed to operate in any one of a number of different structural orientations, such as the cruciform orientation.

Method embodiments are disclosed which are capable of detecting one or more single nucleotide polymorphisms with or without the use of substrates such as encoded microparticles. For instance, a sample comprising or suspected of comprising a target nucleic acid comprising at least one single nucleotide polymorphism may be incubated with at least two label extender probes each comprising a different L-1 sequence, and a label probe system with the sample comprising or suspected of comprising the target nucleic acid comprising the at least one single nucleotide polymorphism, wherein at least one L-1 sequence is complementary to the at least one single nucleotide polymorphism. Finally, the targets may be detected with the label probe system when the label probe system is associated with the sample.

As above, these methods may be applied to various sample types, such as purified chromosomes, tissue culture cells, tissue slices in situ, in cellulo, and in vitro. These methods are also amendable to multiplex applications. The L-1 sequences may also be comprised of nucleotide analogs such as LNA or more specifically, cEt molecules and the like. Due to the inherent flexibility of the present assays, methods, embodiments, compositions and systems, it is shown that the label extenders may be designed to operate in any one of a number of different structural orientations, such as the cruciform orientation.

These general embodiments may also include application to detection of micro-RNA species, such as mature and immature miRNA, siRNA and the like. The methods enable the ability to distinguishably detect various forms and species of miRNA on the pathway to maturity within a cell or tissue. Further siRNA may be detected as a control to determine whether siRNA molecules have penetrated desired cell types, tissue types and/or are hybridizing with the appropriate targets. Mutations in miRNA species may also be detected using these methods and various embodiments of the compositions and systems disclosed herein. Further, multiple different miRNA species or siRNA in a single may be detected simultaneously in a single multiplex assay.

Samples may be obtained from any number of organisms, including bacteria, plants, animals, humans, mice, rats, guinea pigs, monkeys, dogs, cats, fish, anaerobic bacteria, aerobic bacteria, fungi, marine life, and the like. Basically any DNA, RNA or like polymer sequence may be subjected to the present embodiments.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

### BRIEF DESCRIPTION OF THE DRAWINGS

FIG. 1 schematically illustrates a typical standard bDNA assay.

FIG. 2 Panels A-E schematically depict a multiplex nucleic acid detection assay, in which the nucleic acids of interest are captured on distinguishable subsets of microspheres and then detected.

FIG. 3 Panels A-D schematically depict an embodiment of a multiplex nucleic acid detection assay, in which the nucleic acids of interest are captured at selected positions on a solid support and then detected. Panel A shows a top view of the solid support, while Panels B-D show the support in cross-section.

FIG. 4 Panel A schematically depicts a double Z label extender configuration. Panel B schematically depicts a cruciform label extender configuration.

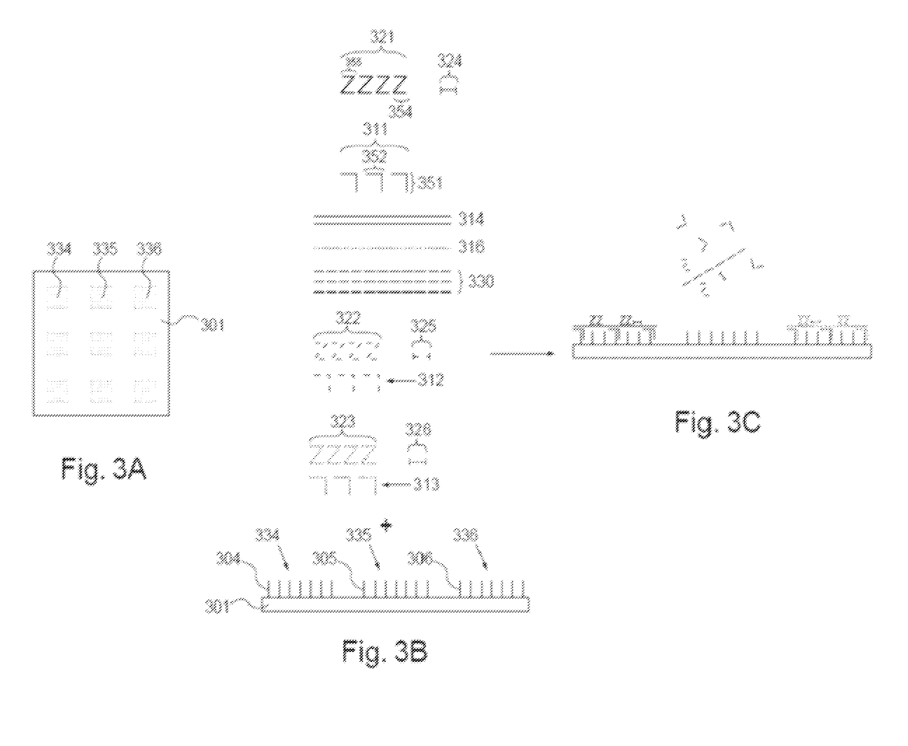

FIG. 5A schematic of amplification multimer complex and labeling system for a cruciform structure label extender design. Note that in this non-limiting depiction, as in others provided herein, only provides a single example of amplifier/pre-amplifier complex. In the assays, more or fewer amplifiers and label probes may be employed as needed.

FIG. 5B schematic of amplification multimer complex and labeling system for a “double z” or ZZ structure label extender design. Note that in this non-limiting depiction, as in others provided herein, only provides a single example of amplifier/pre-amplifier complex. In the assays, more or fewer amplifiers and label probes may be employed as needed.

FIG. 6A depiction of a locked nucleic acid analog known as the constrained ethyl (cEt) nucleic acid analog. Note that as depicted various protecting groups known in the art are presented but may be substituted by any number of suitable protecting groups.

FIG. 6B depiction of a generic locked nucleic acid analog in the β-D, C3-endo, conformation. The letter “B” stands for “base” which may be any one of A, G, C, mC, T or U. The methylene bridge connecting the 2-O atom with the 4-C atom is the chemical structure which “locks” the analog into the energy-favorable β-D conformation. However, it is understood that this bridge may be any number of carbon atoms in length and may contain any number of variable groups or substitutions as has been reported in the literature Note that as depicted various protecting groups known in the art are presented but may be substituted by any number of suitable protecting groups.

FIG. 7A depicts one embodiment of fusion gene or translocation event detection. It is understood that any number of label extender structure variations other than the double Z geometry may be equally suitable and may be utilized for this embodiment.

FIG. 7B depicts one embodiment of fusion gene or translocation event detection. It is understood that any number of label extender structure variations other than the double Z geometry may be equally suitable and may be utilized for this embodiment.

FIG. 8A depiction of single-stranded target SNP detection embodiments utilizing the cruciform (left panel) and the double Z (right panel) structures for the label extenders.

FIG. 8B depiction of double-stranded (dsDNA) target SNP detection embodiments utilizing the cruciform (left panel) and the double Z (right panel) structures for the label extenders.

FIG. 9A depicts various non-limiting conformations and geometries of label extender (LE) probes for detecting single stranded nucleic acid species. Other stereoisomers, conformers and various conformations are possible which achieve similar results but may not be depicted here. Note that for convenience the amplifiers and pre-amplifiers and label probes are not fully represented for all figures. The single line in light shading labeled as “label probe system” is meant to denote all possible configurations of label probe structures as depicted in FIGS. 6A, 6B, 12A and 12B.

FIG. 9B depicts various non-limiting conformations and geometries of label extender (LE) probes for detecting double-stranded nucleic acid species (ability to distinguish between double-stranded DNA targets and ssDNA or RNA targets). Other stereoisomers, conformers and various conformations are possible which achieve similar results but may not be depicted here. Note that for convenience the amplifiers and pre-amplifiers and label probes are not fully represented for all figures. The single line in light shading labeled as “label probe system” is meant to denote all possible configurations of label probe structures as depicted in FIGS. 6A, 6B, 12A and 12B.

FIG. 10 depiction of one embodiment of miRNA detection.

FIGS. 11A and 11B depict directionality of various label extenders and the possibility that label extenders may be designed in either direction as indicated.

FIG. 12 depict one embodiment in which the present assay is capable of distinguishing between mature and immature miRNA species, i.e. pri-miRNA species.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a molecule” includes a plurality of such molecules, and the like.

The term “about” as used herein indicates the value of a given quantity varies by +/−10% of the value, or optionally +/−5% of the value, or in some embodiments, by +/−1% of the value so described.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

The term “analog” in the context of nucleic acid analog is meant to denote any of a number of known nucleic acid analogs such as, but not limited to, LNA, PNA, etc. For instance, it has been reported that LNA, when incorporated into oligonucleotides, exhibit an increase in the duplex melting temperature of 2° C. to 8° C. per analog incorporated into a single strand of the duplex. The melting temperature effect of incorporated analogs may vary depending on the chemical structure of the analog, e.g. the structure of the atoms present in the bridge between the 2-O atom and the 4-C atom of the ribose ring of a nucleic acid.

Various bicyclic nucleic acid analogs have been prepared and reported. (See, for example, Singh et al., Chem. Commun., 1998, 4:455-456; Koshkin et al., Tetrahedron, 1998, 54:3607-3630; Wahlestedt et al., Proc. Natl. Acad. Sci. U.S.A., 2000, 97:5633-5638; Kumar et al., Bioorg. Med. Chem. Lett., 1998, 8:2219-2222; Wengel et al., PCT International Application Number PCT/DK98/00303 which published as WO 99/14226 on Mar. 25, 1999; Singh et al., J. Org. Chem., 1998, 63:10035-10039, the text of each is incorporated by reference herein, in their entirety). Examples of issued US Patents and Published U.S. Patent Applications disclosing various bicyclic nucleic acids include, for example, U.S. Pat. Nos. 6,770,748, 6,268,490 and 6,794,499 and U.S. Patent Application Publication Nos. 20040219565, 20040014959, 20030207841, 20040192918, 20030224377, 20040143114, 20030087230 and 20030082807, the text of each of which is incorporated by reference herein, in their entirety.

Various 5′-modified nucleosides have also been reported. (See, for example: Mikhailov et al., Nucleosides and Nucleotides, 1991, 10:393-343; Saha et al., J. Org. Chem., 1995, 60:788-789; Beigleman et al., Nucleosides and Nucleotides, 1995, 14:901-905; Wang, et al., Bioorganic & Medicinal Chemistry Letters, 1999, 9:885-890; and PCT Internation Application Number WO94/22890 which was published Oct. 13, 1994, the text of each of which is incorporated by reference herein, in their entirety).

Oligonucleotides in solution as single stranded species rotate and move in space in various energy-minimized conformations. Upon binding and ultimately hybridizing to a complementary sequence, an oligonucleotide is known to undergo a conformational transition from the relatively random coil structure of the single stranded state to the ordered structure of the duplex state. With these physical-chemical dynamics in mind, a number of conformationally-restricted oligonucleotides analogs, including bicyclic and tricyclic nucleoside analogues, have been synthesized, incorporated into oligonucleotides and tested for their ability to hybridize. It has been found that various nucleic acid analogs, such as the common “Locked Nucleic Acid” or LNA, exhibit a very low energy-minimized state upon hybridizing to the complementary oligonucleotide, even when the complementary oligonucleotide is wholly comprised of the native or natural nucleic acids A, T, C, U and G.

Other examples of issued US patents and published applications include, but are not limited to: U.S. Pat. Nos. 7,053,207, 6,770,748, 6,268,490 and 6,794,499 and published U.S. applications 20040219565, 20040014959, 20030207841, 20040192918, 20030224377, 20040143114 and 20030082807; the text of each is incorporated by reference herein, in their entirety.

Additionally, bicyclo[3.3.0] nucleosides (bcDNA) with an additional C-3′,C-5′-ethano-bridge have been reported for all five of the native or natural nucleobases (G, A, T, C and U) whereas (C) has been synthesised only with T and A nucleobases. (See, Tarkoy et al., Helv. Chim. Acta, 1993, 76:481; Tarkoy and C. Leumann, Angew. Chem. Int. Ed. Engl., 1993, 32:1432; Egli et al., J. Am. Chem. Soc., 1993, 115:5855; Tarkoy et al., Helv. Chim. Acta, 1994, 77:716; M. Bolli and C. Leumann, Angew. Chem., Int. Ed. Engl., 1995, 34:694; Bolli et al., Helv. Chim. Acta, 1995, 78:2077; Litten et al., Bioorg. Med. Chem. Lett., 1995, 5:1231; J. C. Litten and C. Leumann, Helv. Chim. Acta, 1996, 79:1129; Bolli et al., Chem. Biol., 1996, 3:197; Bolli et al., Nucleic Acids Res., 1996, 24:4660). Oligonucleotides containing these analogues have been found to form Watson-Crick bonded duplexes with complementary DNA and RNA oligonucleotides. The thermostability of the resulting duplexes, however, is varied and not always improved over comparable native hybridized oligonucleotide sequences. All bcDNA oligomers exhibited an increase in sensitivity to the ionic strength of the hybridization media compared to natural counterparts.

A bicyclo[3.3.0] nucleoside dimer containing an additional C-2′,C-3′-dioxalane ring has been reported in the literature having an unmodified nucleoside where the additional ring is part of the internucleoside linkage replacing a natural phosphodiester linkage. As either thymine-thymine or thymine-5-methylcytosine blocks, a 15-mer polypyrimidine sequence containing seven dimeric blocks and having alternating phosphodiester- and riboacetal-linkages exhibited a substantially decreased Tₘ in hybridization with complementary ssRNA as compared to a control sequence with exclusively natural phosphordiester internucleoside linkages. (See, Jones et al., J. Am. Chem. Soc., 1993, 115:9816).

Other U.S. patents have disclosed various modifications of these analogs that exhibit the desired properties of being stably integrated into oligonucleotide sequences and increasing the melting temperature at which hybridization occurs, thus producing a very stable, energy-minimized duplex with oligonucleotides comprising even native nucleic acids. (See, for instance, U.S. Pat. Nos. 7,572,582, 7,399,845, 7,034,133, 6,794,499 and 6,670,461, all of which are incorporated herein by reference in their entirety for all purposes).

For instance, U.S. Pat. No. 7,399,845 provides 6-modified bicyclic nucleosides, oligomeric compounds and compositions prepared therefrom, including novel synthetic intermediates, and methods of preparing the nucleosides, oligomeric compounds, compositions, and novel synthetic intermediates. The '845 patent discloses nucleosides having a bridge between the 4′ and 2′-positions of the ribose portion having the formula: 2′-O—C(H)(Z)-4′ and oligomers and compositions prepared therefrom. In a preferred embodiment, Z is in a particular configuration providing either the (R) or (S) isomer, e.g. 2′-O,4′-methanoribonucleoside. It was shown that this nucleic acid analog exists as the strictly constrained N-conformer 2′-exo-3′-endo conformation. Oligonucleotides of 12 nucleic acids in length have been shown, when comprised completely or partially of the Imanishi et al. analogs, to have substantially increased melting temperatures, showing that the corresponding duplexes with complementary native oligonucleotides are very stable. (See, Imanishi et al., “Synthesis and property of novel conformationally constrained nucleoside and oligonucleotide analogs,” The Sixteenth International Congress of Heterocyclic Chemistry, Aug. 10-15, 1997, incorporated herein by reference in its entirety for all purposes).

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

The “Tₘ” (melting temperature) of a nucleic acid duplex under specified conditions (e.g., relevant assay conditions) is the temperature at which half of the base pairs in a population of the duplex are disassociated and half are associated. The Tₘ for a particular duplex can be calculated and/or measured, e.g., by obtaining a thermal denaturation curve for the duplex (where the Tₘ is the temperature corresponding to the midpoint in the observed transition from double-stranded to single-stranded form).

The term “complementary” refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “capture extender” or “CE” is a polynucleotide that is capable of hybridizing to a nucleic acid of interest and to a capture probe. The capture extender typically has a first polynucleotide sequence C-1, which is complementary to the capture probe, and a second polynucleotide sequence C-3, which is complementary to a polynucleotide sequence of the nucleic acid of interest. Sequences C-1 and C-3 are typically not complementary to each other. The capture extender is preferably single-stranded.

A “capture probe” or “CP” is a polynucleotide that is capable of hybridizing to at least one capture extender and that is tightly bound (e.g., covalently or noncovalently, directly or through a linker, e.g., streptavidin-biotin or the like) to a solid support, a spatially addressable solid support, a slide, a particle, a microsphere, or the like. The capture probe typically comprises at least one polynucleotide sequence C-2 that is complementary to polynucleotide sequence C-1 of at least one capture extender. The capture probe is preferably single-stranded.

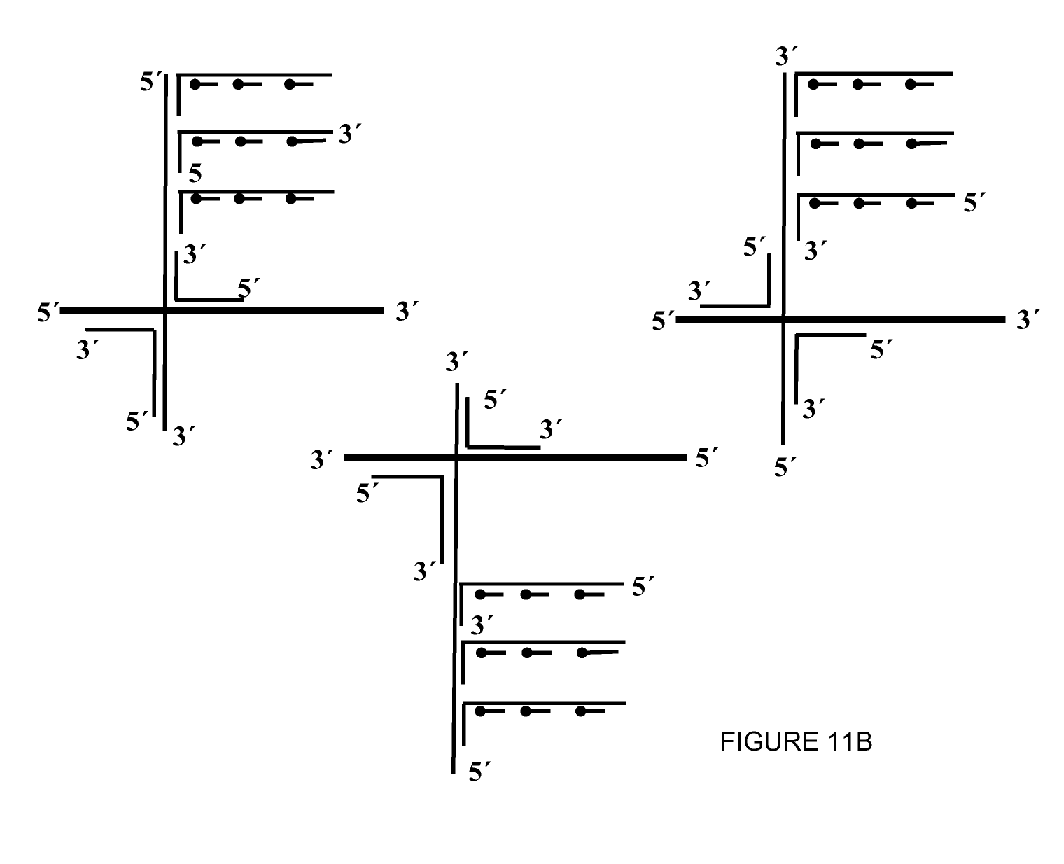

A “label extender” or “LE” is a polynucleotide that is capable of hybridizing to a nucleic acid of interest and to a label probe system. The label extender typically has a first polynucleotide sequence L-1, which is complementary to a polynucleotide sequence of the nucleic acid of interest, and a second polynucleotide sequence L-2, which is complementary to a polynucleotide sequence of the label probe system (e.g., L-2 can be complementary to a polynucleotide sequence of an amplification multimer, a preamplifier, a label probe, or the like). The label extender is preferably single-stranded. Label extenders designed in both directions are contemplated, i.e. a label extender in the 3 to 5 direction could just as easily be designed to bind in the reverse direction as depicted in the Figures. For instance, see FIGS. 11A and 11B for exemplary depictions of the various configurations which may be designed to be suitable for use in the presently disclosed invention.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

A “label probe system” comprises one or more polynucleotides that collectively comprise a label and at least two polynucleotide sequences M-1, each of which is capable of hybridizing to a label extender. The label provides a signal, directly or indirectly. Polynucleotide sequence M-1 is typically complementary to sequence L-2 in the label extenders. The at least two polynucleotide sequences M-1 are optionally identical sequences or different sequences. The label probe system can include a plurality of label probes (e.g., a plurality of identical label probes) and an amplification multimer; it optionally also includes a preamplifier or the like, or optionally includes only label probes, for example.

An “amplification multimer” is a polynucleotide comprising a plurality of polynucleotide sequences M-2, typically (but not necessarily) identical polynucleotide sequences M-2. Polynucleotide sequence M-2 is complementary to a polynucleotide sequence in the label probe. The amplification multimer also includes at least one polynucleotide sequence that is capable of hybridizing to a label extender or to a nucleic acid that hybridizes to the label extender, e.g., a preamplifier. For example, the amplification multimer optionally includes at least one (and preferably at least two) polynucleotide sequence(s) M-1, optionally identical sequences M-1; polynucleotide sequence M-1 is typically complementary to polynucleotide sequence L-2 of the label extenders. Similarly, the amplification multimer optionally includes at least one polynucleotide sequence that is complementary to a polynucleotide sequence in a preamplifier. The amplification multimer can be, e.g., a linear or a branched nucleic acid. That is, the amplification multimer may be entirely comprised of a single contiguous chain of nucleic acids, or alternative a first chain possessing the sequence M-1 and additionally possessing one more sequences A-1 that are complementary to sequences A-2 on separate oligonucleotides which comprise one or more repeats of the sequence M-2. Thus, the amplification multimer may in fact be an assembly of multiple oligonucleotides comprising or consisting of a pre-amplifier possessing the M-2 sequence and one or more A-1 sequences; and one or more amplifier oligonucleotides possessing the sequence A-2 and one or more sequences M-2. Upon hybridization the structure may yield a tree-like geometrical shape comprising a single pre-amplifier, multiple amplifiers and attached to the amplifiers, multiple label probes which hybridize to site(s) M-2. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. No. 5,635,352, U.S. Pat. No. 5,124,246, U.S. Pat. No. 5,710,264, and U.S. Pat. No. 5,849,481.

A “label probe” or “LP” is a single-stranded polynucleotide that comprises a label (or optionally that is configured to bind to a label) that directly or indirectly provides a detectable signal. The label probe typically comprises a polynucleotide sequence that is complementary to the repeating polynucleotide sequence M-2 of the amplification multimer; however, if no amplification multimer is used in the bDNA assay, the label probe can, e.g., hybridize directly to a label extender.

A “preamplifier” is a nucleic acid that serves as an intermediate between one or more label extenders and amplifiers. Typically, the preamplifier is capable of hybridizing simultaneously to at least two label extenders and to a plurality of amplifiers.

A “microsphere” is a small spherical, or roughly spherical, particle. A microsphere typically has a diameter less than about 1000 micrometers (e.g., less than about 100 micrometers, optionally less than about 10 micrometers).

“Microparticles” include particles having a code, including sets of encoded microparticles. (See, for instance, U.S. patent application Ser. No. 11/521,057, allowed, which is incorporated herein by reference in its entirety for all purposes). Such encoded microparticles may have a longest dimension of 50 microns, an outer surface substantially of glass and a spatial code that can be read with optical magnification. A microparticle may be cuboid in shape and elongated along the Y direction in the Cartesian coordinate. The cross-sections perpendicular to the length of the microparticle may have substantially the same topological shape—such as square shape. Microparticles may have a set of segments and gaps intervening the segments in parallel along the axis of the longest dimension if the microparticle is rectangular. Specifically, segments with different lengths (the dimension along the length of the microparticle, e.g. along the Y direction) may represent different coding elements; whereas gaps preferably have the same length for differentiating the segments during detection of the microparticles. The segments of the microparticle may be fully enclosed within the microparticle, i.e. completely encapsulated by a surrounding outer layer which may be silicon/glass. As an alternative feature, the segments can be arranged such that the geometric centers of the segments are aligned to the geometric central axis of the elongated microparticle. A particular sequence of segments and gaps thereby represent a code within each micoparticle. The codes may be derived from a pre-determined coding scheme thereby allowing identification of the microparticle. The microparticles may additionally have various structural aberrations, such as tags or tabs, on one or more ends, thus allowing for a two-fold or more increase in code space. The microparticles may also be present as a “bi-particle” wherein the microparticle actually comprises two or more particles stuck together, i.e. missing the last etching step so as to allow two particles to remain attached together with an intervening material between them comprised of material consistent with the coating present on the rest of the microparticle. (See, for instance, U.S. patent application Ser. No. 12/779,413, filed May 13, 2010, incorporated herein by reference in its entirety for all purposes).

A “microorganism” is an organism of microscopic or submicroscopic size. Examples include, but are not limited to, bacteria, fungi, yeast, protozoans, microscopic algae (e.g., unicellular algae), viruses (which are typically included in this category although they are incapable of growth and reproduction outside of host cells), subviral agents, viroids, and mycoplasma.

A first polynucleotide sequence that is located “5′ of” a second polynucleotide sequence on a nucleic acid strand is positioned closer to the 5′ terminus of the strand than is the second polynucleotide sequence. Similarly, a first polynucleotide sequence that is located “3′ of” a second polynucleotide sequence on a nucleic acid strand is positioned closer to the 3′ terminus of the strand than is the second polynucleotide sequence.

A variety of additional terms are defined or otherwise characterized herein.

### DETAILED DESCRIPTION

The present invention provides methods, compositions, and kits for capture and detection of various types of nucleic acids, particularly multiplex capture and detection. As will be shown in more detail below, the disclosed methodologies and compositions are highly adaptable to many applications. A non-limiting list of various embodiments is as follows:

A general class of embodiments includes methods of capturing two or more nucleic acids of interest and identification thereof. In the methods, a sample, a pooled population of particles (or microparticles, or encoded microparticles), and two or more subsets of n target capture probes, wherein n is at least two, are provided. The sample comprises or is suspected of comprising the nucleic acids of interest. The pooled population of particles includes two or more subsets of particles. The particles in each subset have associated therewith a different capture probes. Each subset of n capture extenders is capable of hybridizing to one of the nucleic acids of interest, and the capture extenders in each subset are capable of hybridizing to one of the capture probes and thereby associating each subset of n target capture probes with a selected subset of the particles. Preferably, a plurality of the particles in each subset is distinguishable from a plurality of the particles in every other subset. (Typically, substantially all of the particles in each subset are distinguishable from substantially all of the particles in every other subset.) Each nucleic acid of interest can thus, by hybridizing to its corresponding subset of n capture extenders which are in turn hybridized to a corresponding capture probes, be associated with an identifiable subset of the particles. Alternatively, the particles in the various subsets need not be distinguishable from each other (for example, in embodiments in which any nucleic acid of interest present is to be isolated, amplified, and/or detected, without regard to its identity, following its capture on the particles.)

In one embodiment of the following methodologies and compositions, a particular nucleic acid of interest, or target oligonucleotide, may be captured to a surface through cooperative hybridization of multiple target capture probes to the nucleic acid. Each of the capture extenders (CE) has a first polynucleotide sequence that can hybridize to the target nucleic acid and a second polynucleotide sequence that can hybridize to a complementary sequence on a capture probe that is bound to a surface. The temperature and the stability of the complex between a single CE and its CP can be controlled such that binding of a single CE to a target nucleic acid and to the CP is not sufficient to stably capture the nucleic acid on the surface to which the CP is bound, whereas simultaneous binding of two or more CEs to a target nucleic acid can capture it on the surface vie the two or more CPs. Assays requiring such cooperative hybridization of multiple target capture probes for capture of each nucleic acid of interest results in high specificity and low background from cross-hybridization of the target capture probes with other, non-target nucleic acids. Such low background and minimal cross-hybridization are typically substantially more difficult to achieve in multiplex than a single-plex capture of nucleic acids, because the number of potential nonspecific interactions are greatly increased in a multiplex experiment due to the increased number of probes used (e.g., the greater number of target capture probes). Requiring multiple simultaneous CE-CP interactions for the capture of a target nucleic acid minimizes the chance that nonspecific capture will occur, even when some nonspecific target-CE and/or CE-CP interactions occur.

Branched-chain DNA (bDNA) signal amplification technology has been used, e.g., to detect and quantify mRNA transcripts in cell lines and to determine viral loads in blood. (See, for instance, Player et al. (2001) “Single-copy gene detection using branched DNA (bDNA) in situ hybridization,” J. Histochem. Cytochem., 49:603-611, Van Cleve et al., Mol. Cell. Probes, (1998) 12:243-247, and U.S. Pat. No. 7,033,758, each of which is incorporated herein by reference in their entirety for all purposes). The bDNA assay is a sandwich nucleic acid hybridization procedure that enables direct measurement of mRNA expression, e.g., from crude cell lysate. It provides direct quantification of nucleic acid molecules at physiological levels. Several advantages of the technology distinguish it from other DNA/RNA amplification technologies, including linear amplification, good sensitivity and dynamic range, great precision/specificity and accuracy, simple sample preparation procedure, and reduced sample-to-sample variation.

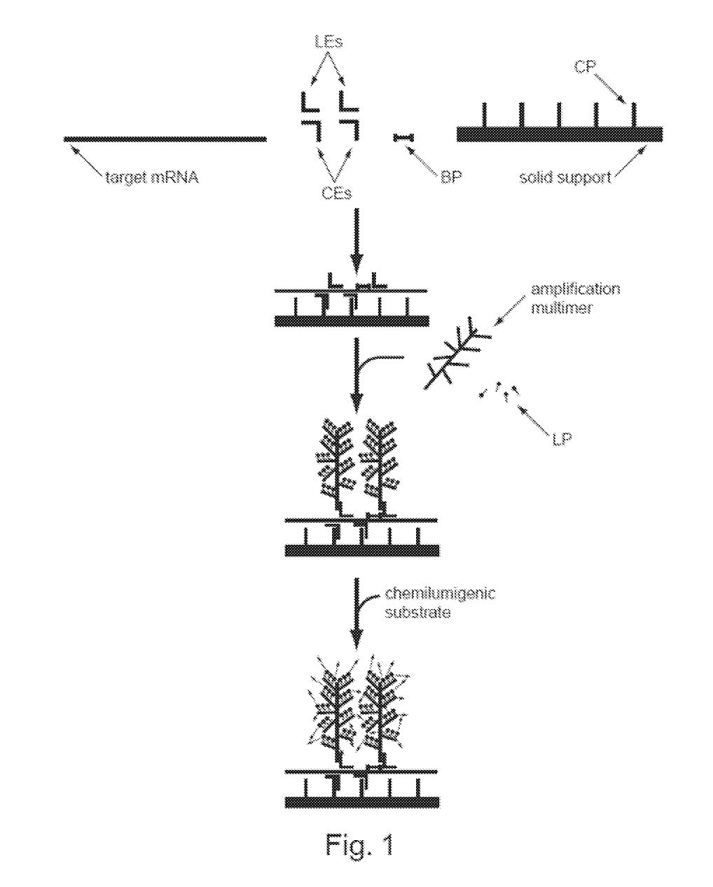

In brief, in a typical bDNA assay for gene expression analysis (FIG. 1), a target mRNA whose expression is to be detected is released from cells and captured by a Capture Probe (CP) on a solid surface (e.g., a well of a microtiter plate) through synthetic oligonucleotide probes called Capture Extenders (CEs). Each capture extender has a first polynucleotide sequence that can hybridize to the target mRNA and a second polynucleotide sequence that can hybridize to the capture probe. Typically, two or more capture extenders are used. Probes of another type, called Label Extenders (LEs), hybridize to different sequences on the target mRNA and to sequences on an amplification multimer. Additionally, Blocking Probes (BPs), which hybridize to regions of the target mRNA not occupied by CEs or LEs, are often used to reduce non-specific target probe binding. A probe set for a given mRNA thus consists of CEs, LEs, and optionally BPs for the target mRNA. The CEs, LEs, and BPs are complementary to nonoverlapping sequences in the target mRNA, and are typically, but not necessarily, contiguous.

Signal amplification begins with the binding of the LEs to the target mRNA. An amplification multimer is then typically hybridized to the LEs. The amplification multimer has multiple copies of a sequence that is complementary to a label probe (it is worth noting that the amplification multimer is typically, but not necessarily, a branched-chain nucleic acid; for example, the amplification multimer can be a branched, forked, or comb-like nucleic acid or a linear nucleic acid). A label, for example, alkaline phosphatase, is covalently attached to each label probe. (Alternatively, the label can be noncovalently bound to the label probes.) In the final step, labeled complexes are detected, e.g., by the alkaline phosphatase-mediated degradation of a chemilumigenic substrate, e.g., dioxetane. Luminescence is reported as relative light unit (RLUs) on a microplate reader. The amount of chemiluminescence is proportional to the level of mRNA expressed from the target gene.

In the preceding example, the amplification multimer and the label probes comprise a label probe system. In another example, the label probe system also comprises a preamplifier, e.g., as described in U.S. Pat. No. 5,635,352 and U.S. Pat. No. 5,681,697, which further amplifies the signal from a single target mRNA. In yet another example, the label extenders hybridize directly to the label probes and no amplification multimer or preamplifier is used, so the signal from a single target mRNA molecule is only amplified by the number of distinct label extenders that hybridize to that mRNA.

Basic bDNA assays have been well described. See, e.g., U.S. Pat. No. 4,868,105 to Urdea et al. entitled “Solution phase nucleic acid sandwich assay”; U.S. Pat. No. 5,635,352 to Urdea et al. entitled “Solution phase nucleic acid sandwich assays having reduced background noise”; U.S. Pat. No. 5,681,697 to Urdea et al. entitled “Solution phase nucleic acid sandwich assays having reduced background noise and kits therefor”; U.S. Pat. No. 5,124,246 to Urdea et al. entitled “Nucleic acid multimers and amplified nucleic acid hybridization assays using same”; U.S. Pat. No. 5,624,802 to Urdea et al. entitled “Nucleic acid multimers and amplified nucleic acid hybridization assays using same”; U.S. Pat. No. 5,849,481 to Urdea et al. entitled “Nucleic acid hybridization assays employing large comb-type branched polynucleotides”; U.S. Pat. No. 5,710,264 to Urdea et al. entitled “Large comb type branched polynucleotides”; U.S. Pat. No. 5,594,118 to Urdea and Horn entitled “Modified N-4 nucleotides for use in amplified nucleic acid hybridization assays”; U.S. Pat. No. 5,093,232 to Urdea and Horn entitled “Nucleic acid probes”; U.S. Pat. No. 4,910,300 to Urdea and Horn entitled “Method for making nucleic acid probes”; U.S. Pat. No. 5,359,100; U.S. Pat. No. 5,571,670; U.S. Pat. No. 5,614,362; U.S. Pat. No. 6,235,465; U.S. Pat. No. 5,712,383; U.S. Pat. No. 5,747,244; U.S. Pat. No. 6,232,462; U.S. Pat. No. 5,681,702; U.S. Pat. No. 5,780,610; U.S. Pat. No. 5,780,227 to Sheridan et al. entitled “Oligonucleotide probe conjugated to a purified hydrophilic alkaline phosphatase and uses thereof”; U.S. patent application Publication No. US2002172950 by Kenny et al. entitled “Highly sensitive gene detection and localization using in situ branched-DNA hybridization”; Wang et al. (1997) “Regulation of insulin preRNA splicing by glucose” Proc Nat Acad Sci USA 94:4360-4365; Collins et al. (1998) “Branched DNA (bDNA) technology for direct quantification of nucleic acids: Design and performance” in Gene Quantification, F Ferre, ed.; and Wilber and Urdea (1998) “Quantification of HCV RNA in clinical specimens by branched DNA (bDNA) technology” Methods in Molecular Medicine: Hepatitis C 19:71-78. In addition, kits for performing basic bDNA assays (QUANTIGENE™ kits, comprising instructions and reagents such as amplification multimers, alkaline phosphatase labeled label probes, chemilumigenic substrate, capture probes immobilized on a solid support, and the like) are commercially available, e.g., from Panomics, Inc. (on the world wide web at (www.panomics.com). General protocols and user's guides on how the QUANTIGENE® system works and explanation of kits and components may be found at the Panomics website (see, www.panomics.com/index.php?id=product_{—}1#product_lit_{—}1). Specifically, user's manual, “QUANTIGENE® 2.0 Reagent System User Manual,” (2007, 32 pages) provided at the Panomics website is incorporated herein by reference in its entirety for all purposes. Software for designing probe sets for a given mRNA target (i.e., for designing the regions of the CEs, LEs, and optionally BPs that are complementary to the target) is also commercially available (e.g., ProbeDesigner™ from Panomics, Inc.; see also Bushnell et al. (1999) “ProbeDesigner: for the design of probe sets for branched DNA (bDNA) signal amplification assays Bioinformatics 15:348-55).

The basic bDNA assay, however, permits detection of only a single target nucleic acid per assay, while, as described above, detection of multiple nucleic acids is frequently desirable.

Among other aspects, the present invention provides multiplex bDNA assays that can be used for simultaneous detection of two or more target nucleic acids. Similarly, one aspect of the present invention provides bDNA assays, singleplex or multiplex, that have reduced background from nonspecific hybridization events.

Among other aspects, the present invention provides a multiplex bDNA assay that can be used for simultaneous detection of two or more target nucleic acids. The assay temperature and the stability of the complex between a single CE and its corresponding CP can be controlled such that binding of a single CE to a nucleic acid and to the CP is not sufficient to stably capture the nucleic acid on the surface to which the CP is bound, whereas simultaneous binding of two or more CEs to a nucleic acid can capture it on the surface. Requiring such cooperative hybridization of multiple CEs for capture of each nucleic acid of interest results in high specificity and low background from cross-hybridization of the CEs with other, non-target nucleic acids. For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of CEs and LEs). Requiring multiple simultaneous CE-CP interactions for the capture of a target nucleic acid minimizes the chance that nonspecific capture will occur, even when some nonspecific CE-CP interactions do occur.

In general, in the assays of the invention, two or more label extenders are used to capture a single component of the label probe system (e.g., a preamplifier or amplification multimer). The assay temperature and the stability of the complex between a single LE and the component of the label probe system (e.g., the preamplifier or amplification multimer) can be controlled such that binding of a single LE to the component is not sufficient to stably associate the component with a nucleic acid to which the LE is bound, whereas simultaneous binding of two or more LEs to the component can capture it to the nucleic acid. Requiring such cooperative hybridization of multiple LEs for association of the label probe system with the nucleic acid(s) of interest results in high specificity and low background from cross-hybridization of the LEs with other, non-target nucleic acids.

For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of CEs and LEs). Requiring multiple simultaneous LE-label probe system component interactions for the capture of the label probe system to a target nucleic acid minimizes the chance that nonspecific capture will occur, even when some nonspecific CE-LE or LE-CP interactions, for example, do occur. This reduction in background through minimization of undesirable cross-hybridization events thus facilitates multiplex detection of the nucleic acids of interest.

The methods of the invention can be used, for example, for multiplex detection of two or more nucleic acids simultaneously, from even complex samples, without requiring prior purification of the nucleic acids, when the nucleic acids are present at low concentration, and/or in the presence of other, highly similar nucleic acids. In one aspect, the methods involve capture of the nucleic acids to particles (e.g., distinguishable subsets of microspheres), while in another aspect, the nucleic acids are captured to a spatially addressable solid support. Compositions, kits, and systems related to the methods are also provided.

### Methods, in General

As noted, one aspect of the invention provides multiplex nucleic acid assays. Thus, one general class of embodiments includes methods of detecting two or more nucleic acids of interest. In one embodiment of the method, a sample comprising or suspected of comprising the nucleic acids of interest, two or more subsets of m label extenders, wherein m is at least two, and a label probe system are provided. Each subset of m label extenders is capable of hybridizing to one of the nucleic acids of interest. The label probe system comprises a label, and a component of the label probe system is capable of hybridizing simultaneously to at least two of the m label extenders in a subset.

Those nucleic acids of interest present in the sample are captured on a solid support. Each nucleic acid of interest captured on the solid support is hybridized to its corresponding subset of m label extenders, and the label probe system is hybridized to the m label extenders. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

In another embodiment, a sample, a pooled population of particles, and two or more subsets of n capture extenders, wherein n is at least two, are provided. The sample comprises or is suspected of comprising the nucleic acids of interest. The pooled population of particles includes two or more subsets of particles, and a plurality of the particles in each subset are distinguishable from a plurality of the particles in every other subset. (Typically, substantially all of the particles in each subset are distinguishable from substantially all of the particles in every other subset.) The particles in each subset have associated therewith a different capture probe. Each subset of n capture extenders is capable of hybridizing to one of the nucleic acids of interest, and the capture extenders in each subset are capable of hybridizing to one of the capture probes and thereby associating each subset of n capture extenders with a selected subset of the particles. Each nucleic acid of interest can thus, by hybridizing to its corresponding subset of n capture extenders which are in turn hybridized to a corresponding capture probe, be associated with an identifiable subset of the particles.

Essentially any suitable solid support can be employed in the methods. For example, the solid support can comprise particles such as microspheres or microparticles, or it can comprise a substantially planar and/or spatially addressable support. Different nucleic acids are optionally captured on different distinguishable subsets of particles or at different positions on a spatially addressable solid support. The nucleic acids of interest can be captured to the solid support by any of a variety of techniques, for example, by binding directly to the solid support or by binding to a moiety bound to the support, or through hybridization to another nucleic acid bound to the solid support. Preferably, the nucleic acids are captured to the solid support through hybridization with capture extenders and capture probes.

In one class of embodiments, a pooled population of particles which constitute the solid support is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture probe.

Two or more subsets of n capture extenders, wherein n is at least two, are also provided. Each subset of n capture extenders is capable of hybridizing to one of the nucleic acids of interest, and the capture extenders in each subset are capable of hybridizing to one of the capture probes, thereby associating each subset of n capture extenders with a selected subset of the particles. Each of the nucleic acids of interest present in the sample is hybridized to its corresponding subset of n capture extenders and the subset of n capture extenders is hybridized to its corresponding capture probe, thereby capturing the nucleic acid on the subset of particles with which the capture extenders are associated.

Typically, in this class of embodiments, at least a portion of the particles from each subset are identified and the presence or absence of the label on those particles is detected. Since a correlation exists between a particular subset of particles and a particular nucleic acid of interest, which subsets of particles have the label present indicates which of the nucleic acids of interest were present in the sample.

Essentially any suitable particles, e.g., particles having distinguishable characteristics and to which capture probes can be attached, can be used. For example, in one preferred class of embodiments, the particles are microspheres. The microspheres of each subset can be distinguishable from those of the other subsets, e.g., on the basis of their fluorescent emission spectrum, their diameter, or a combination thereof. For example, the microspheres of each subset can be labeled with a unique fluorescent dye or mixture of such dyes, quantum dots with distinguishable emission spectra, and/or the like. As another example, the particles of each subset can be identified by an optical barcode, unique to that subset, present on the particles.

The particles optionally have additional desirable characteristics. For example, the particles can be magnetic or paramagnetic, which provides a convenient means for separating the particles from solution, e.g., to simplify separation of the particles from any materials not bound to the particles.

In other embodiments, the nucleic acids are captured at different positions on a non-particulate, spatially addressable solid support. Thus, in one class of embodiments, the solid support comprises two or more capture probes, wherein each capture probe is provided at a selected position on the solid support. Two or more subsets of n capture extenders, wherein n is at least two, are provided. Each subset of n capture extenders is capable of hybridizing to one of the nucleic acids of interest, and the capture extenders in each subset are capable of hybridizing to one of the capture probes, thereby associating each subset of n capture extenders with a selected position on the solid support. Each of the nucleic acids of interest present in the sample is hybridized to its corresponding subset of n capture extenders and the subset of n capture extenders is hybridized to its corresponding capture probe, thereby capturing the nucleic acid on the solid support at the selected position with which the capture extenders are associated.

Typically, in this class of embodiments, the presence or absence of the label at the selected positions on the solid support is detected. Since a correlation exists between a particular position on the support and a particular nucleic acid of interest, which positions have a label present indicates which of the nucleic acids of interest were present in the sample.

The solid support typically has a planar surface and is typically rigid, but essentially any spatially addressable solid support can be adapted to the practice of the present invention. Exemplary materials for the solid support include, but are not limited to, glass, silicon, silica, quartz, plastic, polystyrene, nylon, and nitrocellulose. As just one example, an array of capture probes can be formed at selected positions on a glass slide as the solid support.

In any of the embodiments described herein in which capture extenders are utilized to capture the nucleic acids to the solid support, n, the number of capture extenders in a subset, is at least one, preferably at least two, and more preferably at least three. n can be at least four or at least five or more. Typically, but not necessarily, n is at most ten. For example, n can be between three and ten, e.g., between five and ten or between five and seven, inclusive. Use of fewer capture extenders can be advantageous, for example, in embodiments in which nucleic acids of interest are to be specifically detected from samples including other nucleic acids with sequences very similar to that of the nucleic acids of interest. In other embodiments (e.g., embodiments in which capture of as much of the nucleic acid as possible is desired), however, n can be more than 10, e.g., between 20 and 50. n can be the same for all of the subsets of capture extenders, but it need not be; for example, one subset can include three capture extenders while another subset includes five capture extenders. The n capture extenders in a subset preferably hybridize to nonoverlapping polynucleotide sequences in the corresponding nucleic acid of interest. The nonoverlapping polynucleotide sequences can, but need not be, consecutive within the nucleic acid of interest.

Each capture extender is capable of hybridizing to its corresponding capture probe. The capture extender typically includes a polynucleotide sequence C-1 that is complementary to a polynucleotide sequence C-2 in its corresponding capture probe. Capture of the nucleic acids of interest via hybridization to the capture extenders and capture probes optionally involves cooperative hybridization. In one aspect, the capture extenders and capture probes are configured as described in U.S. patent application 60/680,976 filed May 12, 2005 by Luo et al., entitled “Multiplex branched-chain DNA assays.” In one aspect, C-1 and C-2 are 20 nucleotides or less in length. In one class of embodiments, C-1 and C-2 are between 9 and 17 nucleotides in length (inclusive), preferably between 12 and 15 nucleotides (inclusive). For example, C-1 and C-2 can be 14, 15, 16, or 17 nucleotides in length, or they can be between 9 and 13 nucleotides in length (e.g., for lower hybridization temperatures, e.g., hybridization at room temperature).

The capture probe can include polynucleotide sequence in addition to C-2, or C-2 can comprise the entire polynucleotide sequence of the capture probe. For example, each capture probe optionally includes a linker sequence between the site of attachment of the capture probe to the particles and sequence C-2 (e.g., a linker sequence containing 8 Ts, as just one possible example).

It will be evident that the amount of overlap between each individual capture extender and its corresponding capture probe (i.e., the length of C-1 and C-2) affects the Tₘ, of the complex between that capture extender and capture probe, as does, e.g., the GC base content of sequences C-1 and C-2. Typically, all the capture probes are the same length (as are sequences C-1 and C-2) from subset of particles to subset, but not necessarily so. Depending, e.g., on the precise nucleotide sequence of C-2, different support capture probes optionally have different lengths and/or different length sequences C-2, to achieve the desired Tₘ. Different support capture probe-target capture probe complexes optionally have the same or different Tₘs.

It will also be evident that the number of capture extenders required for stable capture of a nucleic acid depends, in part, on the amount of overlap between the capture extenders and the capture probe (i.e., the length of C-1 and C-2). For example, if n is 5-7 for a 14 nucleotide overlap, n could be 3-5 for a 15 nucleotide overlap or 2-3 for a 16 nucleotide overlap.

As noted, the hybridizing the subset of n capture extenders to the corresponding support capture probe is performed at a hybridization temperature which is greater than a melting temperature Tₘ, of a complex between each individual capture extender and its corresponding capture probe. The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the T.

Stable capture of nucleic acids of interest, e.g., while minimizing capture of extraneous nucleic acids (e.g., those to which n−1 or fewer of the target capture probes bind) can be achieved, for example, by balancing n (the number of target capture probes), the amount of overlap between the capture extenders and the capture probes (the length of C-1 and C-2), and/or the stringency of the conditions under which the target capture probes, the nucleic acids, and the support capture probes are hybridized.

Appropriate combinations of n, amount of complementarity between the capture extenders and the capture probes, and stringency of hybridization can, for example, be determined experimentally by one of skill in the art. For example, a particular value of n and a particular set of hybridization conditions can be selected, while the number of nucleotides of complementarity between the capture extenders and the capture probes is varied until hybridization of the n capture extenders to a nucleic acid captures the nucleic acid while hybridization of a single capture extender does not efficiently capture the nucleic acid. Similarly, n, amount of complementarity, and stringency of hybridization can be selected such that the desired nucleic acid of interest is captured while other nucleic acids present in the sample are not efficiently captured. Stringency can be controlled, for example, by controlling the formamide concentration, chaotropic salt concentration, salt concentration, pH, organic solvent content, and/or hybridization temperature.

For a given nucleic acid of interest, the corresponding target capture probes are preferably complementary to physically distinct, nonoverlapping sequences in the nucleic acid of interest, which are preferably, but not necessarily, contiguous. The Tₘs of the individual capture extender-nucleic acid complexes are preferably greater than the hybridization temperature, e.g., by 5° C. or 10° C. or preferably by 15° C. or more, such that these complexes are stable at the hybridization temperature. Sequence C-3, which is the sequence of the CE which is complementary to the target nucleic acid, for each capture extender is typically (but not necessarily) about 17-35 nucleotides in length, with about 30-70% GC content. Potential capture extender sequences (e.g., potential sequences C-3) are optionally examined for possible interactions with non-corresponding nucleic acids of interest, repetitive sequences (such as polyC or polyT, for example), any detection probes used to detect the nucleic acids of interest, and/or any relevant genomic sequences, for example; sequences expected to cross-hybridize with undesired nucleic acids are typically not selected for use in the target support capture probes. Examination can be, e.g., visual (e.g., visual examination for complementarity), computational (e.g., computation and comparison of percent sequence identity and/or binding free energies; for example, sequence comparisons can be performed using BLAST software publicly available through the National Center for Biotechnology Information on the world wide web at ncbi.nlm.nih.gov), and/or experimental (e.g., cross-hybridization experiments). Capture probe sequences are preferably similarly examined, to ensure that the polynucleotide sequence C-1 complementary to a particular capture probe's sequence C-2 is not expected to cross-hybridize with any of the other capture probes that are to be associated with other subsets of particles.

The methods are useful for multiplex detection of nucleic acids, optionally highly multiplex detection. Thus, the two or more nucleic acids of interest (i.e., the nucleic acids to be detected) optionally comprise five or more, 10 or more, 20 or more, 30 or more, 40 or more, 50 or more, or even 100 or more nucleic acids of interest, while the two or more subsets of m label extenders comprise five or more, 10 or more, 20 or more, 30 or more, 40 or more, 50 or more, or even 100 or more subsets of m label extenders. In embodiments in which capture extenders, particulate solid supports, and/or spatially addressable solid support are used, a like number of subsets of capture extenders, subsets of particles, and/or selected positions on the solid support are provided.

The label probe system optionally includes an amplification multimer and a plurality of label probes, wherein the amplification multimer is capable of hybridizing to the label extenders and to a plurality of label probes. In another aspect, the label probe system includes a preamplifier, a plurality of amplification multimers, and a plurality of label probes, wherein the preamplifier hybridizes to the label extenders, and the amplification multimers hybridize to the preamplifier and to the plurality of label probes. As another example, the label probe system can include only label probes, which hybridize directly to the label extenders. In one class of embodiments, the label probe comprises the label, e.g., a covalently attached label. In other embodiments, the label probe is configured to bind a label; for example, a biotinylated label probe can bind to a streptavidin-associated label.

The label can be essentially any convenient label that directly or indirectly provides a detectable signal. In one aspect, the label is a fluorescent label (e.g., a fluorophore or quantum dot). Detecting the presence of the label on the particles thus comprises detecting a fluorescent signal from the label. In embodiments in which the solid support comprises particles, fluorescent emission by the label is typically distinguishable from any fluorescent emission by the particles, e.g., microspheres, and many suitable fluorescent label-fluorescent microsphere combinations are possible. As other examples, the label can be a luminescent label, a light-scattering label (e.g., colloidal gold particles), or an enzyme (e.g., HRP). Various labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), ATTO dyes (Sigma-Aldrich, St. Louis, Mo.), or any other suitable label.

As noted above, a component of the label probe system is capable of hybridizing simultaneously to at least two of the m label extenders in a subset. Typically, the component of the label probe system that hybridizes to the two or more label extenders is an amplification multimer or preamplifier. Preferably, binding of a single label extender to the component of the label probe system (e.g., the amplification multimer or preamplifier) is insufficient to capture the label probe system to the nucleic acid of interest to which the label extender binds. Thus, in one aspect, the label probe system comprises an amplification multimer or preamplifier, which amplification multimer or preamplifier is capable of hybridizing to the at least two label extenders, and the label probe system (or the component thereof) is hybridized to the m label extenders at a hybridization temperature, which hybridization temperature is greater than a melting temperature Tₘ of a complex between each individual label extender and the amplification multimer or preamplifier. The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the T. It is worth noting that the hybridization temperature can be the same or different than the temperature at which the label extenders and optional capture extenders are hybridized to the nucleic acids of interest.

Each label extender typically includes a polynucleotide sequence L-1 that is complementary to a polynucleotide sequence in the corresponding nucleic acid of interest and a polynucleotide sequence L-2 that is complementary to a polynucleotide sequence in the component of the label probe system (e.g., the preamplifier or amplification multimer). It will be evident that the amount of overlap between each individual label extender and the component of the label probe system (i.e., the length of L-2 and M-1) affects the Tₘ of the complex between the label extender and the component, as does, e.g., the GC base content of sequences L-2 and M-1. Optionally, all the label extenders have the same length sequence L-2 and/or identical polynucleotide sequences L-2. Alternatively, different label extenders can have different length and/or sequence polynucleotide sequences L-2. It will also be evident that the number of label extenders required for stable capture of the component to the nucleic acid of interest depends, in part, on the amount of overlap between the label extenders and the component (i.e., the length of L-2 and M-1).

Stable capture of the component of the label probe system by the at least two label extenders, e.g., while minimizing capture of extraneous nucleic acids, can be achieved, for example, by balancing the number of label extenders that bind to the component, the amount of overlap between the label extenders and the component (the length of L-2 and M-1), and/or the stringency of the conditions under which the label extenders and the component are hybridized. For instance, when detecting a large message RNA of several hundred base pairs or less, any number of label extenders may be used, such as, for instance, 1-30 pairs of label extender probes, or 2-28 pairs of label extender probes, or 3-25 pairs of label extender probes, or 4-20 pairs of label extender probes, or a number of label extender probe pairs which is suitable to specifically attach the label probe system to the target with the desired affinity.

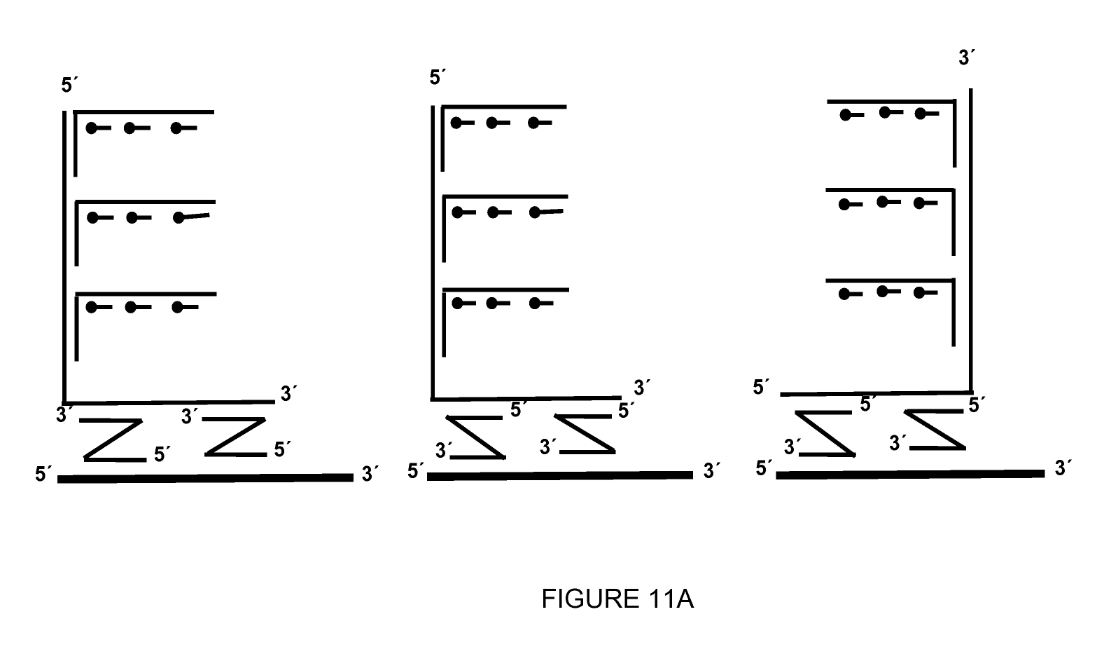

As noted above, while some embodiments generally utilize two label extender probes to hybridize to each pre-amplifier, it is possible in other embodiments to design systems in which three label extender probes hybridize to a single target and single pre-amplifier probe, or even four label extender probes per pre-amplifier. Further, when the target nucleic acid is particularly short, as in siRNA or miRNA, it is possible to use only a single label extender probe, in concert with a single capture extender probe, to detect the target. (See, for instance, FIG. 10). Alternatively, if performing the assay in situ, for example, or in other suitable conditions, a single pair of label extender probes may be designed to contain the entire complement to the target sequence (half of which would be encoded in the L-1 sequence of a first label extender probe, and the other half of which would be encoded in the second L-1 sequence of the second label extender probe).

Appropriate combinations of the amount of complementarity between the label extenders and the component of the label probe system, number of label extenders binding to the component, and stringency of hybridization can, for example, be determined experimentally by one of skill in the art. For example, a particular number of label extenders and a particular set of hybridization conditions can be selected, while the number of nucleotides of complementarity between the label extenders and the component is varied until hybridization of the label extenders to a nucleic acid captures the component to the nucleic acid while hybridization of a single label extender does not efficiently capture the component. Stringency can be controlled, for example, by controlling the formamide concentration, chaotropic salt concentration, salt concentration, pH, organic solvent content, and/or hybridization temperature.

As noted, the Tₘ of any nucleic acid duplex can be directly measured, using techniques well known in the art. For example, a thermal denaturation curve can be obtained for the duplex, the midpoint of which corresponds to the T. It will be evident that such denaturation curves can be obtained under conditions having essentially any relevant pH, salt concentration, solvent content, and/or the like.

The Tₘ for a particular duplex (e.g., an approximate Tₘ) can also be calculated. For example, the Tₘ for an oligonucleotide-target duplex can be estimated using the following algorithm, which incorporates nearest neighbor thermodynamic parameters: Tm (Kelvin)=ΔH°/(ΔS°+R ln Cₜ), where the changes in standard enthalpy (ΔH°) and entropy (ΔS°) are calculated from nearest neighbor thermodynamic parameters (see, e.g., SantaLucia (1998) “A unified view of polymer, dumbbell, and oligonucleotide DNA nearest-neighbor thermodynamics” Proc. Natl. Acad. Sci. USA 95:1460-1465, Sugimoto et al. (1996) “Improved thermodynamic parameters and helix initiation factor to predict stability of DNA duplexes” Nucleic Acids Research 24: 4501-4505, Sugimoto et al. (1995) “Thermodynamic parameters to predict stability of RNA/DNA hybrid duplexes” Biochemistry 34:11211-11216, and et al. (1998) “Thermodynamic parameters for an expanded nearest-neighbor model for formation of RNA duplexes with Watson-Crick base pairs” Biochemistry 37: 14719-14735), R is the ideal gas constant (1.987 cal·K⁻¹mole⁻¹), and Cₜ is the molar concentration of the oligonucleotide. The calculated Tₘ is optionally corrected for salt concentration, e.g., Na⁺ concentration, using the formula 1/Tₘ(Na⁺)=1/Tₘ(1M)+(4.29f (G·C)−3.95)×10⁻⁵ ln [Na⁺]+9.40×10⁻⁶ ln²[Na⁺]. See, e.g., Owczarzy et al. (2004) “Effects of Sodium Ions on DNA Duplex Oligomers: Improved Predictions of Melting Temperatures” Biochemistry 43:3537-3554 for further details. A Web calculator for estimating Tₘ using the above algorithms is available on the Internet at scitools.idtdna.com/analyzer/oligocalc.asp. Other algorithms for calculating Tₘ are known in the art and are optionally applied to the present invention.

Typically, the component of the label probe system (e.g., the amplification multimer or preamplifier) is capable of hybridizing simultaneously to two of the m label extenders in a subset, although it optionally hybridizes to three, four, or more of the label extenders. In one class of embodiments, e.g., embodiments in which two (or more) label extenders bind to the component of the label probe system, sequence L-2 is 20 nucleotides or less in length. For example, L-2 can be between 9 and 17 nucleotides in length, e.g., between 12 and 15 nucleotides in length, between 13 and 15 nucleotides in length, or between 13 and 14 nucleotides in length. As noted, m is at least two, and can be at least three, at least five, at least 10, or more. m can be the same or different from subset to subset of label extenders.

The label extenders can be configured in any of a variety ways. For example, the two label extenders that hybridize to the component of the label probe system can assume a cruciform arrangement, with one label extender having L-1 5′ of L-2 and the other label extender having L-1 3′ of L-2. Unexpectedly, however, a configuration in which either the 5′ or the 3′ end of both label extenders hybridizes to the nucleic acid while the other end binds to the component yields stronger binding of the component to the nucleic acid than does a cruciform arrangement of the label extenders. Thus, in one class of embodiments, the at least two label extenders (e.g., the m label extenders in a subset) each have L-1 5′ of L-2 or each have L-1 3′ of L-2. For example, L-1, which hybridizes to the nucleic acid of interest, can be at the 5′ end of each label extender, while L-2, which hybridizes to the component of the label probe system, is at the 3′ end of each label extender (or vice versa). L-1 and L-2 are optionally separated by additional sequence. In one exemplary embodiment, L-1 is located at the 5′ end of the label extender and is about 20-30 nucleotides in length, L-2 is located at the 3′ end of the label extender and is about 13-14 nucleotides in length, and L-1 and L-2 are separated by a spacer (e.g., 5 Ts).

A label extender, preamplifier, amplification multimer, label probe, capture probe and/or capture extender optionally comprises at least one non-natural nucleotide. For example, a label extender and the component of the label probe system (e.g., the amplification multimer or preamplifier) optionally comprise, at complementary positions, at least one pair of non-natural nucleotides that base pair with each other but that do not Watson-Crick base pair with the bases typical to biological DNA or RNA (i.e., A, C, G, T, or U). Examples of normatural nucleotides include, but are not limited to, Locked NucleicAcid™ nucleotides (available from Exiqon A/S, (www.) exiqon.com; see, e.g., SantaLucia Jr. (1998) Proc Natl Acad Sci 95:1460-1465) and isoG, isoC, and other nucleotides used in the AEGIS system (Artificially Expanded Genetic Information System, available from EraGen Biosciences, (www.) eragen.com; see, e.g., U.S. Pat. No. 6,001,983, U.S. Pat. No. 6,037,120, and U.S. Pat. No. 6,140,496). Use of such non-natural base pairs (e.g., isoG-isoC base pairs) in the probes can, for example, reduce background and/or simplify probe design by decreasing cross hybridization, or it can permit use of shorter probes (e.g., shorter sequences L-2 and M-1) when the non-natural base pairs have higher binding affinities than do natural base pairs.

The methods can optionally be used to quantitate the amounts of the nucleic acids of interest present in the sample. For example, in one class of embodiments, an intensity of a signal from the label is measured, e.g., for each subset of particles or selected position on the solid support, and correlated with a quantity of the corresponding nucleic acid of interest present.

As noted, blocking probes are optionally also hybridized to the nucleic acids of interest, which can reduce background in the assay. For a given nucleic acid of interest, the corresponding label extenders, optional capture extenders, and optional blocking probes are preferably complementary to physically distinct, nonoverlapping sequences in the nucleic acid of interest, which are preferably, but not necessarily, contiguous. The Tₘs of the capture extender-nucleic acid, label extender-nucleic acid, and blocking probe-nucleic acid complexes are preferably greater than the temperature at which the capture extenders, label extenders, and/or blocking probes are hybridized to the nucleic acid, e.g., by 5° C. or 10° C. or preferably by 15° C. or more, such that these complexes are stable at that temperature. Potential CE and LE sequences (e.g., potential sequences C-3 and L-1) are optionally examined for possible interactions with non-corresponding nucleic acids of interest, LEs or CEs, the preamplifier, the amplification multimer, the label probe, and/or any relevant genomic sequences, for example; sequences expected to cross-hybridize with undesired nucleic acids are typically not selected for use in the CEs or LEs. See, e.g., Player et al. (2001) “Single-copy gene detection using branched DNA (bDNA) in situ hybridization” J Histochem Cytochem 49:603-611 and U.S. patent application 60/680,976. Examination can be, e.g., visual (e.g., visual examination for complementarity), computational (e.g., computation and comparison of binding free energies), and/or experimental (e.g., cross-hybridization experiments). Capture probe sequences are preferably similarly examined, to ensure that the polynucleotide sequence C-1 complementary to a particular capture probe's sequence C-2 is not expected to cross-hybridize with any of the other capture probes that are to be associated with other subsets of particles or selected positions on the support.

At any of various steps, materials not captured on the solid support are optionally separated from the support. For example, after the capture extenders, nucleic acids, label extenders, blocking probes, and support-bound capture probes are hybridized, the support is optionally washed to remove unbound nucleic acids and probes; after the label extenders and amplification multimer are hybridized, the support is optionally washed to remove unbound amplification multimer; and/or after the label probes are hybridized to the amplification multimer, the support is optionally washed to remove unbound label probe prior to detection of the label.

In embodiments in which different nucleic acids are captured to different subsets of particles, one or more of the subsets of particles is optionally isolated, whereby the associated nucleic acid of interest is isolated. Similarly, nucleic acids can be isolated from selected positions on a spatially addressable solid support. The isolated nucleic acid can optionally be removed from the particles and/or subjected to further manipulation, if desired (e.g., amplification by PCR or the like).

As another exemplary embodiment, determining which subsets of particles have a nucleic acid of interest captured on the particles may further comprise amplifying any nucleic acid of interest captured on the particles. A wide variety of techniques for amplifying nucleic acids are known in the art, including, but not limited to, PCR (polymerase chain reaction), rolling circle amplification, and transcription mediated amplification. (See, e.g., Hatch et al. (1999) “Rolling circle amplification of DNA immobilized on solid surfaces and its application to multiplex mutation detection” Genet Anal. 15:35-40; Baner et al. (1998) “Signal amplification of padlock probes by rolling circle replication,” Nucleic Acids Res., 26:5073-8; and Nallur et al. (2001) “Signal amplification by rolling circle amplification on DNA microarrays,” Nucleic Acids Res., 29:E118.) A labeled primer and/or labeled nucleotides are optionally incorporated during amplification. In other embodiments, the nucleic acids of interest captured on the particles are detected and/or amplified without identifying the subsets of particles and/or the nucleic acids (e.g., in embodiments in which the subsets of particles are not distinguishable).

The methods can be used to detect the presence of the nucleic acids of interest in essentially any type of sample. For example, the sample can be derived from an animal, a human, a plant, a cultured cell, a virus, a bacterium, a pathogen, and/or a microorganism. The sample optionally includes a cell lysate, an intercellular fluid, a bodily fluid (including, but not limited to, blood, serum, saliva, urine, sputum, or spinal fluid), and/or a conditioned culture medium, and is optionally derived from a tissue (e.g., a tissue homogenate), a biopsy, and/or a tumor. Similarly, the nucleic acids can be essentially any desired nucleic acids (e.g., DNA, RNA, mRNA, rRNA, miRNA, etc.). As just a few examples, the nucleic acids of interest can be derived from one or more of an animal, a human, a plant, a cultured cell, a microorganism, a virus, a bacterium, or a pathogen.

Due to cooperative hybridization of multiple target capture probes to a nucleic acid of interest, for example, even nucleic acids present at low concentration can be captured. Thus, in one class of embodiments, at least one of the nucleic acids of interest is present in the sample in a non-zero amount of 200 attomole (amol) or less, 150 amol or less, 100 amol or less, 50 amol or less, 10 amol or less, 1 amol or less, or even 0.1 amol or less, 0.01 amol or less, 0.001 amol or less, or 0.0001 amol or less. Similarly, two nucleic acids of interest can be captured simultaneously, even when they differ in concentration by 1000-fold or more in the sample. The methods are thus extremely versatile.

Capture of a particular nucleic acid is optionally quantitative. Thus, in one exemplary class of embodiments, the sample includes a first nucleic acid of interest, and at least 30%, at least 50%, at least 80%, at least 90%, at least 95%, or even at least 99% of a total amount of the first nucleic acid present in the sample is captured on a first subset of particles. Second, third, etc. nucleic acids can similarly be quantitatively captured. Such quantitative capture can occur without capture of a significant amount of undesired nucleic acids, even those of very similar sequence to the nucleic acid of interest.

As noted, the methods can be used for gene expression analysis. Accordingly, in one class of embodiments, the two or more nucleic acids of interest comprise two or more mRNAs. The methods can also be used for clinical diagnosis and/or detection of microorganisms, e.g., pathogens. Thus, in certain embodiments, the nucleic acids include bacterial and/or viral genomic RNA and/or DNA (double-stranded or single-stranded), plasmid or other extra-genomic DNA, or other nucleic acids derived from microorganisms (pathogenic or otherwise). It will be evident that double-stranded nucleic acids of interest will typically be denatured before hybridization with capture extenders, label extenders, and the like.

The methods may similarly be applied towards detection and identification of single nucleotide polymorphisms (SNPs) residing in a genomic sample. The methods are very flexible and can be applied equally as well to SNP detection across the entire genome, if desired. Various methods of SNP detection may be employed, as explained in further detail below.

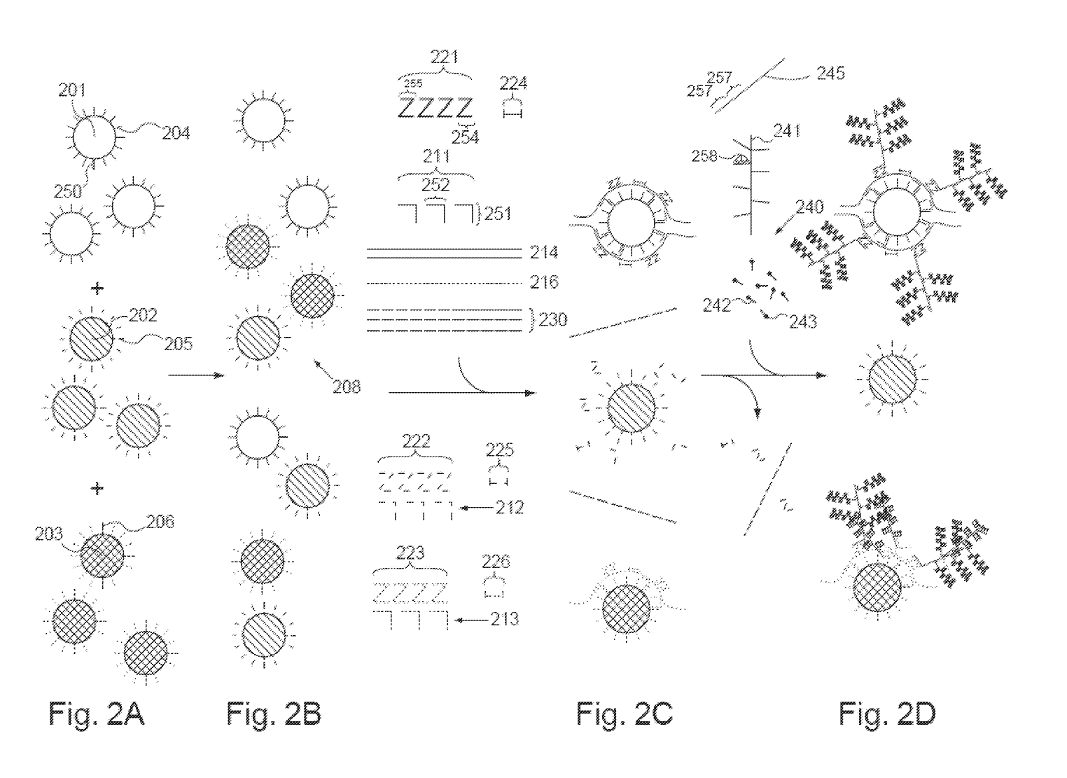

An exemplary embodiment is schematically illustrated in FIG. 2. Panel A illustrates three distinguishable subsets of microspheres 201, 202, and 203, which have associated therewith capture probes 204, 205, and 206, respectively. Each capture probe includes a sequence C-2 (250), which is different from subset to subset of microspheres. The three subsets of microspheres are combined to form pooled population 208 (Panel B). A subset of capture extenders is provided for each nucleic acid of interest; subset 211 for nucleic acid 214, subset 212 for nucleic acid 215 which is not present, and subset 213 for nucleic acid 216. Each capture extender includes sequences C-1 (251, complementary to the respective capture probe's sequence C-2) and C-3 (252, complementary to a sequence in the corresponding nucleic acid of interest). Three subsets of label extenders (221, 222, and 223 for nucleic acids 214, 215, and 216, respectively) and three subsets of blocking probes (224, 225, and 226 for nucleic acids 214, 215, and 216, respectively) are also provided. Each label extender includes sequences L-1 (254, complementary to a sequence in the corresponding nucleic acid of interest) and L-2 (255, complementary to M-1). Non-target nucleic acids 230 are also present in the sample of nucleic acids.

Subsets of label extenders 221 and 223 are hybridized to nucleic acids 214 and 216, respectively. In addition, nucleic acids 214 and 216 are hybridized to their corresponding subset of capture extenders (211 and 213, respectively), and the capture extenders are hybridized to the corresponding capture probes (204 and 206, respectively), capturing nucleic acids 214 and 216 on microspheres 201 and 203, respectively (Panel C). Materials not bound to the microspheres (e.g., capture extenders 212, nucleic acids 230, etc.) are separated from the microspheres by washing. Label probe system 240 including preamplifier 245 (which includes two sequences M-1 257), amplification multimer 241 (which includes sequences M-2 258), and label probe 242 (which contains label 243) is provided. Each preamplifier 245 is hybridized to two label extenders, amplification multimers 241 are hybridized to the preamplifier, and label probes 242 are hybridized to the amplification multimers (Panel D). Materials not captured on the microspheres are optionally removed by washing the microspheres. Microspheres from each subset are identified, e.g., by their fluorescent emission spectrum (λ₂ and λ₃, Panel E), and the presence or absence of the label on each subset of microspheres is detected (λ₁, Panel E). Since each nucleic acid of interest is associated with a distinct subset of microspheres, the presence of the label on a given subset of microspheres correlates with the presence of the corresponding nucleic acid in the original sample.

As depicted in FIG. 2, all of the label extenders in all of the subsets typically include an identical sequence L-2. Optionally, however, different label extenders (e.g., label extenders in different subsets) can include different sequences L-2. Also as depicted in FIG. 2, each capture probe typically includes a single sequence C-2 and thus hybridizes to a single capture extender. Optionally, however, a capture probe can include two or more sequences C-2 and hybridize to two or more capture extenders. Similarly, as depicted, each of the capture extenders in a particular subset typically includes an identical sequence C-1, and thus only a single capture probe is needed for each subset of particles; however, different capture extenders within a subset optionally include different sequences C-1 (and thus hybridize to different sequences C-2, within a single capture probe or different capture probes on the surface of the corresponding subset of particles).

In the embodiment depicted in FIG. 2, the label probe system includes the preamplifier, amplification multimer, and label probe. It will be evident that similar considerations apply to embodiments in which the label probe system includes only an amplification multimer and label probe or only a label probe.

The various hybridization and capture steps can be performed simultaneously or sequentially, in any convenient order. For example, in embodiments in which capture extenders are employed, each nucleic acid of interest can be hybridized simultaneously with its corresponding subset of m label extenders and its corresponding subset of n capture extenders, and then the capture extenders can be hybridized with capture probes associated with the solid support. Materials not captured on the support are preferably removed, e.g., by washing the support, and then the label probe system is hybridized to the label extenders.

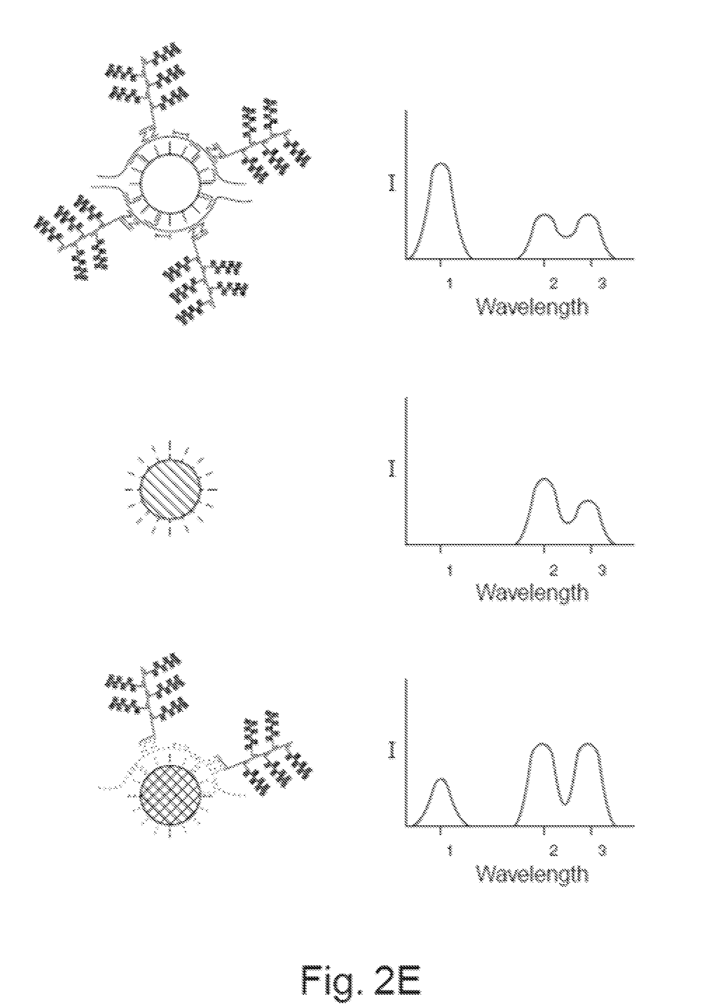

Another exemplary embodiment is schematically illustrated in FIG. 3. Panel A depicts solid support 301 having nine capture probes provided on it at nine selected positions (e.g., 334-336). Panel B depicts a cross section of solid support 301, with distinct capture probes 304, 305, and 306 at different selected positions on the support (334, 335, and 336, respectively). A subset of capture extenders is provided for each nucleic acid of interest. Only three subsets are depicted; subset 311 for nucleic acid 314, subset 312 for nucleic acid 315 which is not present, and subset 313 for nucleic acid 316. Each capture extender includes sequences C-1 (351, complementary to the respective capture probe's sequence C-2) and C-3 (352, complementary to a sequence in the corresponding nucleic acid of interest). Three subsets of label extenders (321, 322, and 323 for nucleic acids 314, 315, and 316, respectively) and three subsets of blocking probes (324, 325, and 326 for nucleic acids 314, 315, and 316, respectively) are also depicted (although nine would be provided, one for each nucleic acid of interest). Each label extender includes sequences L-1 (354, complementary to a sequence in the corresponding nucleic acid of interest) and L-2 (355, complementary to M-1). Non-target nucleic acids 330 are also present in the sample of nucleic acids.

Subsets of label extenders 321 and 323 are hybridized to nucleic acids 314 and 316, respectively. Nucleic acids 314 and 316 are hybridized to their corresponding subset of capture extenders (311 and 313, respectively), and the capture extenders are hybridized to the corresponding capture probes (304 and 306, respectively), capturing nucleic acids 314 and 316 at selected positions 334 and 336, respectively (Panel C). Materials not bound to the solid support (e.g., capture extenders 312, nucleic acids 330, etc.) are separated from the support by washing. Label probe system 340 including preamplifier 345 (which includes two sequences M-1 357), amplification multimer 341 (which includes sequences M-2 358) and label probe 342 (which contains label 343) is provided. Each preamplifier 345 is hybridized to two label extenders, amplification multimers 341 are hybridized to the preamplifier, and label probes 342 are hybridized to the amplification multimers (Panel D). Materials not captured on the solid support are optionally removed by washing the support, and the presence or absence of the label at each position on the solid support is detected. Since each nucleic acid of interest is associated with a distinct position on the support, the presence of the label at a given position on the support correlates with the presence of the corresponding nucleic acid in the original sample.

Another general class of embodiments provides methods of detecting one or more nucleic acids, using the novel label extender configuration described above. In the methods, a sample comprising or suspected of comprising the nucleic acids of interest, one or more subsets of m label extenders, wherein m is at least two, and a label probe system are provided. Each subset of m label extenders is capable of hybridizing to one of the nucleic acids of interest. The label probe system comprises a label, and a component of the label probe system (e.g., a preamplifier or an amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

Those nucleic acids of interest present in the sample are captured on a solid support. Each nucleic acid of interest captured on the solid support is hybridized to its corresponding subset of m label extenders, and the label probe system (or the component thereof) is hybridized to the m label extenders at a hybridization temperature. The hybridization temperature is greater than a melting temperature Tₘ of a complex between each individual label extender and the component of the label probe system. The presence or absence of the label on the solid support is then detected. Since the label is associated with the nucleic acid(s) of interest via hybridization of the label extenders and label probe system, the presence or absence of the label on the solid support is correlated with the presence or absence of the nucleic acid(s) of interest on the solid support and thus in the original sample.

As previously mentioned, the solid support may be one or more particles, microparticles or nanoparticles. The microparticle may be a cuboid structure elongated along the Y direction in the Cartesian coordinate. The cross-sections perpendicular to the length of the microparticle have substantially the same topological shape—which is square in this example. The microparticle may have a set of segments and gaps intervening the segments. Specifically, segments with different lengths (the dimension along the length of the microparticle, e.g. along the Y direction) represent different coding elements; whereas gaps preferably have the same length for differentiating the segments during detection of the microparticles. The segments of the microparticle may be fully enclosed within the microparticle. As an alternative feature, the segments can be arranged such that the geometric centers of the segments are aligned to the geometric central axis of the elongated microparticle. A particular sequence of segments and gaps represents a code. The codes are derived from a pre-determined coding scheme.

Segments of the microparticle can be any suitable form. For instance, each segment of the microparticle may have a substantially square cross-section (i.e. the cross-section in the X-Z plane of a Cartesian coordinate) taken perpendicular to the length (i.e. along the Y direction in the Cartesian coordinate) of the microparticle. The segments may or may not be fabricated to have substantially square cross-section. Other shapes, such as rectangular, circular, and elliptical, jagged, curved or other shapes are also applicable. In particular, the code elements—i.e. segments and gaps, may also take any other suitable desired shape. For example, the segment (and/or the gaps) each may have a cross-section that is rectangular (e.g. with the aspect ratio of the rectangular being 2:1 or higher, such as 4:1 or higher, 10:1 or higher, 20:1 or higher, or even 100:1 or higher, but preferably less than 500:1). The code elements, i.e. the segments and gaps, may take any desired dimensions. As an example, each coding structure may have a characteristic dimension that is 5 μm (microns) or less, such as 3 microns or less, and more preferably 1 micron or less, such as 0.8 or 0.5 microns or less. In particular, when gaps are kept substantially the same dimension while the segments vary in dimension, each gap preferably has a characteristic dimension that is 1.5 microns or less, such as 0.8 or 0.5 microns or less. As one example, if forming the microparticles on a 12-inch silicon wafer with 0.13 line widths, the gap areas can be made to have 0.13 μm minimum widths, with the less transparent segments having widths of from 0.13 μm to much larger (depending upon the desired length of the particle and the encoding scheme and code space desired). Minimum gap widths, as well as minimum segment widths, of from 0.13 to 1.85 μm (e.g. from 0.25 to 0.85 μm) are possible depending upon the wafer fabrication used. Of course larger minimum gap and segment lengths (e.g. 1.85 to 5.0 μm, or more) are also possible. Other sized wafers (4 inch, 6 inch, 8 inch etc.) can of course be used, as well as wafers other than silicon (e.g. glass), as well as other substrates other than silicon (larger glass panels, for example).

The microparticle can have any suitable number of coding structures depending upon the shape or length of the particle, and the code space desired. Specifically, the total number of coding structures of a microparticle can be from 1 to 20, or more typically from 3 to 15, and more typically from 3 to 8. The desired code can be incorporated in and represented by the microparticle in many ways. As an example, the coding elements of the pre-determined coding scheme can be represented by the segment(s)—e.g. segments of different lengths represent different coding elements of the coding scheme. Different spatial arrangements of the segments with the different (or the same) lengths and intervened by gaps represent different codes. In this code-incorporation method, the intervening gaps preferably have substantially the same dimension, especially the length in the direction to which the segments are aligned. As another example, the codes are incorporated in the microparticle by arranging gaps that vary in lengths; while the segments have substantially the same dimension and are disposed between adjacent gaps. In another example, the both segments and gaps vary in their dimensions so as to represent a code. In fact, the code can also be represented in many other alternative ways using the segments, gaps, and the combination thereof. The particle code space may be further expanded by manufacturing a subset of the microparticles such that a tab protrudes from a face of the particle. Further, the code may also incorporate refractive or reflective coatings to expand the maximum number of allowable codes.

To enable detection of codes incorporated in microparticles, the segments and gaps in each microparticle can be composed of materials of different optical, electrical, magnetic, fluid dynamic, or other desired properties that are compatible with the desired detection methods. In one example the segments and gaps are directly spatially distinguishable under transmitted and/or reflected light in the visible spectrum. For example, when the code detection relies upon optical imaging, the distinguishable property (segments vs. gaps) can be a difference in transmissivity to the particular light used for imaging (which can be any desired electromagnetic radiation—e.g. visible and near-visible light, IR, and ultra-violet light. The segments can be made to be more light absorbing (or light reflecting) than the intervening spacing material (or vice versa). Regardless of which specific property is relied upon, the segments and gaps are preferred to exhibit sufficient difference in the specific property such that the difference is detectable using the corresponding code detection method. In particular, when the code is to be detected by means of optical imaging, the segments and gaps are composed of materials exhibiting different transmissivity (in an optical transmittance mode) or reflectivity (in optical reflectance mode) to the specific light used in imaging the microparticles. For example, the segments of the microparticle of the less transparent material can block and/or reflect 30% or more, preferably 50% or more, or e.g. 80% or more, of the visible light or near visible light incident thereon.

The microparticles may be made of organic and/or inorganic materials or a combination of organic and inorganic material. Specifically, the gaps (which are preferably more transmissive to visible or near-visible light) and segments (which are preferably less transmissive to visible or near-visible light as compared to gaps) each can be composed organic or inorganic materials, or a hybrid organic-inorganic material. The segments can be composed of a metal (e.g. aluminum), an early transition metal (e.g. tungsten, chromium, titanium, tantalum or molybdenum), or a metalloid (e.g. silicon or germanium), or combinations (or nitrides, oxides and/or carbides) thereof. In particular, the segments can be composed of a ceramic compound, such as a compound that comprises an oxide of a metalloid or early transition metal, a nitride of a metalloid or early transition metal, or a carbide of a metalloid or early transition metal. Early transition metals are those from columns 3b Sc, Y, Lu, Lr), 4b (Ti, Zr, Hf, Rf), 5b (V, Nb, Ta, Db), 6b (Cr, Mo, W, Sg) and 7b (Mn, Tc, Re, Bh) of the periodic table. However, preferred are early transition metals in columns 4b to 6b, in particular tungsten, titanium, zirconium, hafnium, niobium, tantalum, vanadium and chromium. Alternatively, the particles may be entirely comprised of different forms of silica, glass, or suitable known polymeric materials. The gaps which are in this example more transparent, can comprise any suitable material that is more transparent than the segments. The spacing material can be a siloxane, siloxene or silsesquioxane material, among others, if a hybrid material is selected. The spacing material, if inorganic, can be a glass material. Thin film deposited silicon dioxide is a suitable material, with or without boron or phosphorous doping/alloying agents. Other inorganic glass materials are also suitable such as silicon nitride, silicon oxynitride, germanium oxide, germanium oxynitride, germanium-silicon-oxynitride, or various transition metal oxides for example. A spin on glass (SOG) could also be used. If an organic material is used for the gap material, a plastic (e.g. polystyrene or latex for example) could be used. Both the segments and the gaps can be deposited by any suitable methods such as CVD (chemical vapor deposition), PVD (physical vapor deposition), spin-on, sol gel, etc. If a CVD deposition method is used, the CVD could be LPCVD (low pressure chemical vapor deposition), PECVD (plasma enhanced chemical vapor deposition), APCVD (atmospheric pressure chemical vapor deposition), SACVD (sub atmospheric chemical vapor deposition), etc. If a PVD method is used, sputtering or reactive sputtering are possible depending upon the desired final material. Spin on material (SOG or hybrid organic-inorganic siloxane materials

Other aspects of the microparticles are disclosed in the specification of U.S. patent application Ser. No. 11/521,057, especially at, for instance, sections entitled “Frabrication”, “Detection,” “Method for Producing Codes,” “Coding Scheme,” “Assays,” “A Bioassay Process Using the Microparticles,” and Figures, etc., all of which is incorporated herein by reference for all purposes.

Typically, the one or more nucleic acids of interest comprise two or more nucleic acids of interest, and the one or more subsets of m label extenders comprise two or more subsets of m label extenders.

In one class of embodiments in which the one or more nucleic acids of interest comprise two or more nucleic acids of interest and the one or more subsets of m label extenders comprise two or more subsets of m label extenders, a pooled population of particles which constitute the solid support is provided. The population comprises two or more subsets of particles, and a plurality of the particles in each subset is distinguishable from a plurality of the particles in every other subset. (Typically, substantially all of the particles in each subset are distinguishable from substantially all of the particles in every other subset.) The particles in each subset have associated therewith a different capture probe.

Two or more subsets of n capture extenders, wherein n is at least two, are also provided. Each subset of n capture extenders is capable of hybridizing to one of the nucleic acids of interest, and the capture extenders in each subset are capable of hybridizing to one of the capture probes, thereby associating each subset of n capture extenders with a selected subset of the particles. Each of the nucleic acids of interest present in the sample is hybridized to its corresponding subset of n capture extenders and the subset of n capture extenders is hybridized to its corresponding capture probe, thereby capturing the nucleic acid on the subset of particles with which the capture extenders are associated.

Typically, in this class of embodiments, at least a portion of the particles from each subset are identified and the presence or absence of the label on those particles is detected. Since a correlation exists between a particular subset of particles and a particular nucleic acid of interest, which subsets of particles have the label present indicates which of the nucleic acids of interest were present in the sample.

In other embodiments in which the one or more nucleic acids of interest comprise two or more nucleic acids of interest and the one or more subsets of m label extenders comprise two or more subsets of m label extenders, the nucleic acids are captured at different positions on a non-particulate, spatially addressable solid support. Thus, in one class of embodiments, the solid support comprises two or more capture probes, wherein each capture probe is provided at a selected position on the solid support. Two or more subsets of n capture extenders, wherein n is at least two, are provided. Each subset of n capture extenders is capable of hybridizing to one of the nucleic acids of interest, and the capture extenders in each subset are capable of hybridizing to one of the capture probes, thereby associating each subset of n capture extenders with a selected position on the solid support. Each of the nucleic acids of interest present in the sample is hybridized to its corresponding subset of n capture extenders and the subset of n capture extenders is hybridized to its corresponding capture probe, thereby capturing the nucleic acid on the solid support at the selected position with which the capture extenders are associated.

Typically, in this class of embodiments, the presence or absence of the label at the selected positions on the solid support is detected. Since a correlation exists between a particular position on the support and a particular nucleic acid of interest, which positions have a label present indicates which of the nucleic acids of interest were present in the sample.

Essentially all of the features noted for the methods above apply to these embodiments as well, as relevant; for example, with respect to composition of the label probe system; type of label; type of solid support; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

In one aspect, the invention provides methods for capturing a labeled probe to a target nucleic acid, through hybridization of the labeled probe directly to label extenders hybridized to the nucleic acid or through hybridization of the labeled probe to one or more nucleic acids that are in turn hybridized to the label extenders.

Accordingly, one general class of embodiments provides methods of capturing a label to a first nucleic acid of interest in a multiplex assay in which two or more nucleic acids of interest are to be detected. In the methods, a sample comprising the first nucleic acid of interest and also comprising or suspected of comprising one or more other nucleic acids of interest is provided. A first subset of m label extenders, wherein m is at least two, and a label probe system comprising the label are also provided. The first subset of m label extenders is capable of hybridizing to the first nucleic acid of interest, and a component of the label probe system is capable of hybridizing simultaneously to at least two of the m label extenders in the first subset. The first nucleic acid of interest is hybridized to the first subset of m label extenders, and the label probe system is hybridized to the m label extenders, thereby capturing the label to the first nucleic acid of interest.

Essentially all of the features noted for the embodiments above apply to these methods as well, as relevant; for example, with respect to configuration of the label extenders, number of label extenders per subset, composition of the label probe system, type of label, number of nucleic acids of interest, source of the sample and/or nucleic acids, and/or the like. For example, in one class of embodiments, the label probe system comprises a label probe, which label probe comprises the label, and which label probe is capable of hybridizing simultaneously to at least two of the m label extenders. In other embodiments, the label probe system includes the label probe and an amplification multimer that is capable of hybridizing simultaneously to at least two of the m label extenders. Similarly, in yet other embodiments, the label probe system includes the label probe, an amplification multimer, and a preamplifier that is capable of hybridizing simultaneously to at least two of the m label extenders.

Another general class of embodiments provides methods of capturing a label to a nucleic acid of interest. In the methods, m label extenders, wherein m is at least two, are provided. The m label extenders are capable of hybridizing to the nucleic acid of interest. A label probe system comprising the label is also provided. A component of the label probe system is capable of hybridizing simultaneously to at least two of the m label extenders. Each label extender comprises a polynucleotide sequence L-1 that is complementary to a polynucleotide sequence in the nucleic acid of interest and a polynucleotide sequence L-2 that is complementary to a polynucleotide sequence in the component of the label probe system, and the m label extenders each have L-1 5′ of L-2 or wherein the m label extenders each have L-1 3′ of L-2. The nucleic acid of interest is hybridized to the m label extenders, and the label probe system is hybridized to the m label extenders at a hybridization temperature, thereby capturing the label to the nucleic acid of interest. Preferably, the hybridization temperature is greater than a melting temperature Tₘ of a complex between each individual label extender and the component of the label probe system.

Essentially all of the features noted for the embodiments above apply to these methods as well, as relevant; for example, with respect to configuration of the label extenders, number of label extenders per subset, composition of the label probe system, type of label, and/or the like. For example, in one class of embodiments, the label probe system comprises a label probe, which label probe comprises the label, and which label probe is capable of hybridizing simultaneously to at least two of the m label extenders. In other embodiments, the label probe system includes the label probe and an amplification multimer that is capable of hybridizing simultaneously to at least two of the m label extenders. Similarly, in yet other embodiments, the label probe system includes the label probe, an amplification multimer, and a preamplifier that is capable of hybridizing simultaneously to at least two of the m label extenders.

### EXEMPLARY EMBODIMENTS OF METHODS

### A. Single Nucleotide Polymorphism or Other Target Nucleic Acid Detection

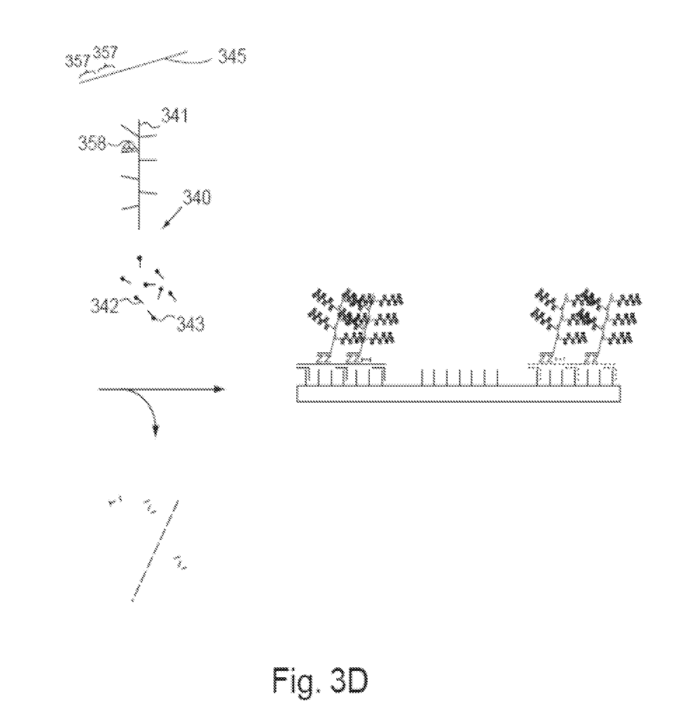

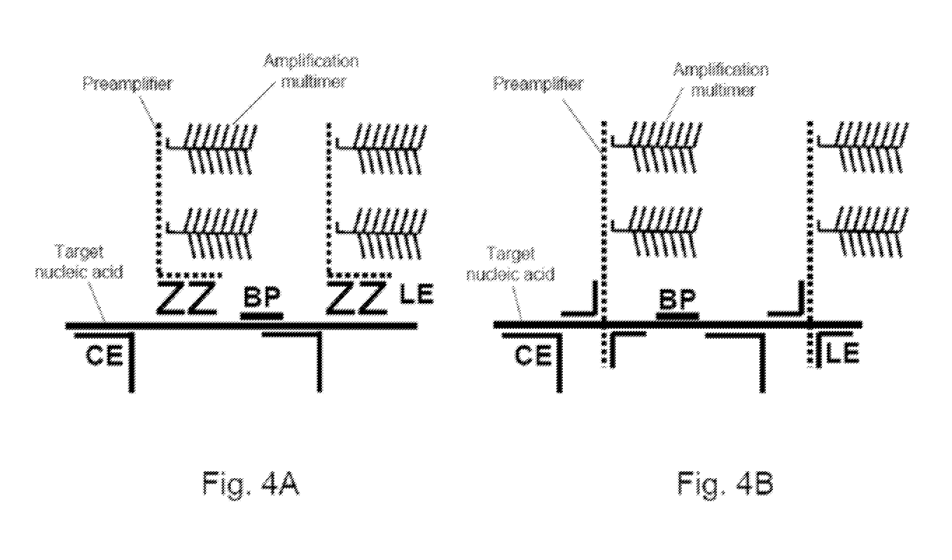

An exemplary method involves the use of multiple technologies to achieve an unparalleled result in the research and diagnostic fields. In this embodiment of the present methods, capture probes and capture extenders are not utilized when the methods and compositions are used to detect targets in vitro or in situ. Label extenders are employed to capture the target nucleic acid and branched DNA technology is used, comprising pre-amplifiers, amplifiers and label probes, to amplify the signal associated with the captured target nucleic acids. (See, for instance, FIGS. 5A and 5B). To make the assay more robust, nucleic acid analogs may be utilized in the capture extender probes. This provides increase specificity for the target, especially in cases where the target is a SNP.

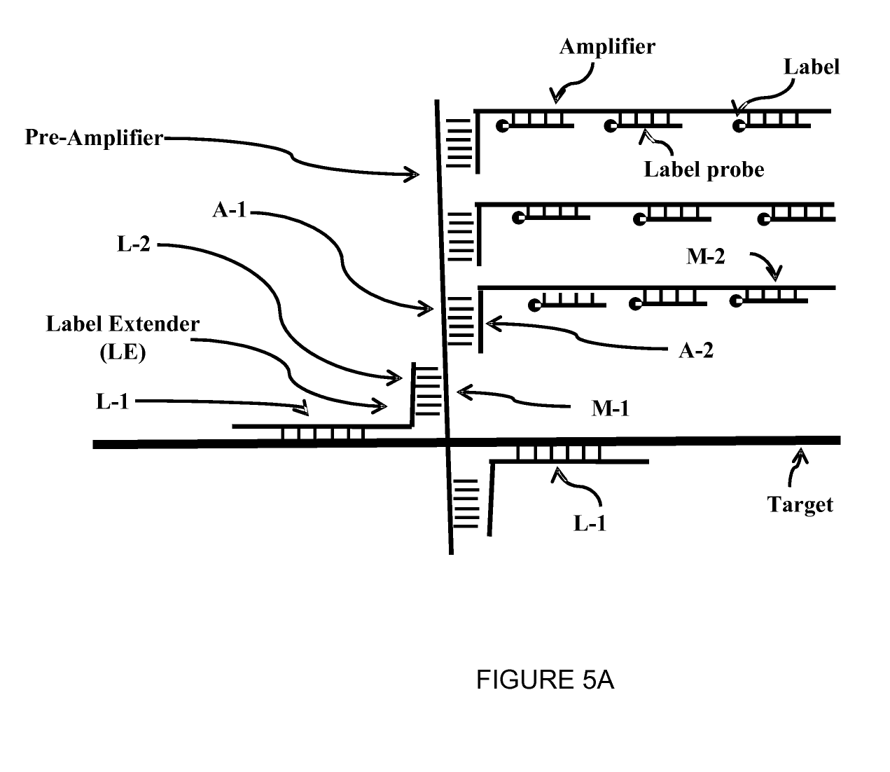

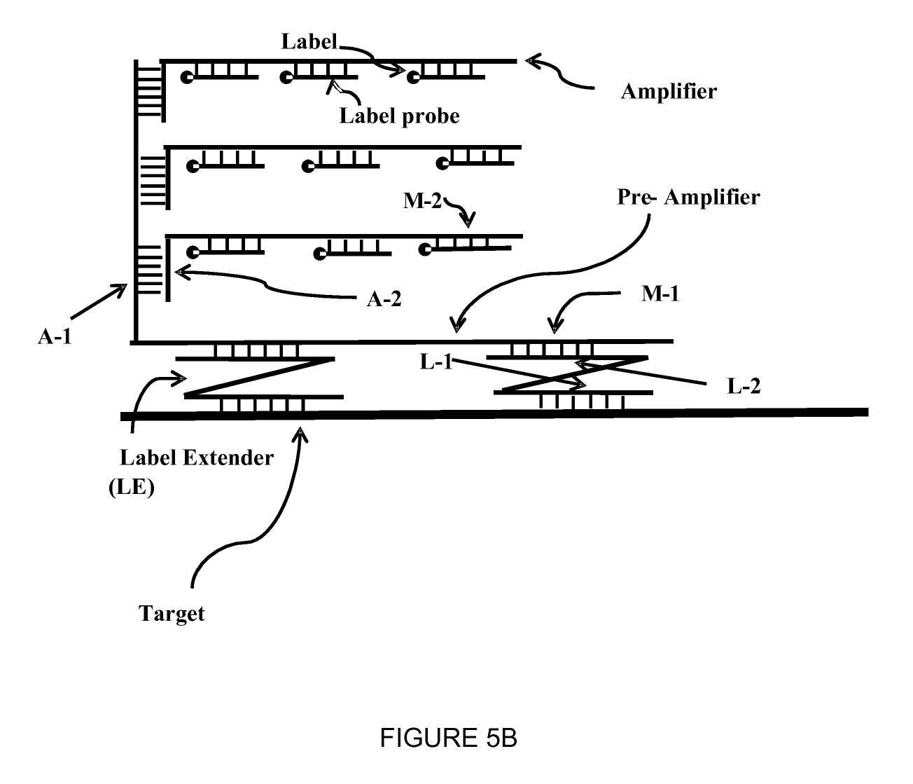

For instance, nucleic acid analogs such as constrained-ethyl (cEt) analogs may be used, as depicted in FIG. 6A. (See, for additional variations of this analog which may also be suitable in the present embodiments, Seth et al., “Short Antisense Oligonucleotides with Novel 2′-4′ Conformationaly Restricted Nucleoside Analogues Show Improved Potency Without Increased Cytotoxicity in Animals,” J. Med. Chem., 52(1):10-13, 2009, incorporated herein by reference in its entirety for all purposes). The capture extender probe may be entirely comprised of such cEt analogs, or may be only partially comprised of cEt analogs. Specifically, the capture extender probe may only have cEt analogs at sequence L-1. The capture extender may have cEt analogs at the C-2 sequence as well as the L-1 sequence and/or cEt content beyond those sequences up to and including the entire capture extender probe. Use of the cEt analogs in the capture portion of the assay is especially beneficial because it is known that cEt analogs, when present in probes, act to increase the melting temperature of the resulting hybridized probe:target pair, which provides increased stability of the hybridized pair and therefore increased stability of the captured target nucleic acid bound to the encoded microparticle.

The length of label extender probes may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Further, the desired melting temperature may also be target-dependant. That is, if a specific miRNA or SNP target is rich in, or has a high content of, G and C bases, then perhaps less melting temperature-increasing nucleic acid analogs, like cEt, will be necessary to achieve the desired melting temperature, as compared to a target region which is rich in A and T bases. In summary, design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

In these assays, cooperative hybridization is employed to bind the pre-amplifier structure to the target using at least two label extender probes per target. FIGS. 5A and 5B depict a typical interaction for such an assay method. In practice, the number of label extenders hybridized per target nucleic acid may be as many as 2, 3, 4, 5, 6, 7 or even 8.

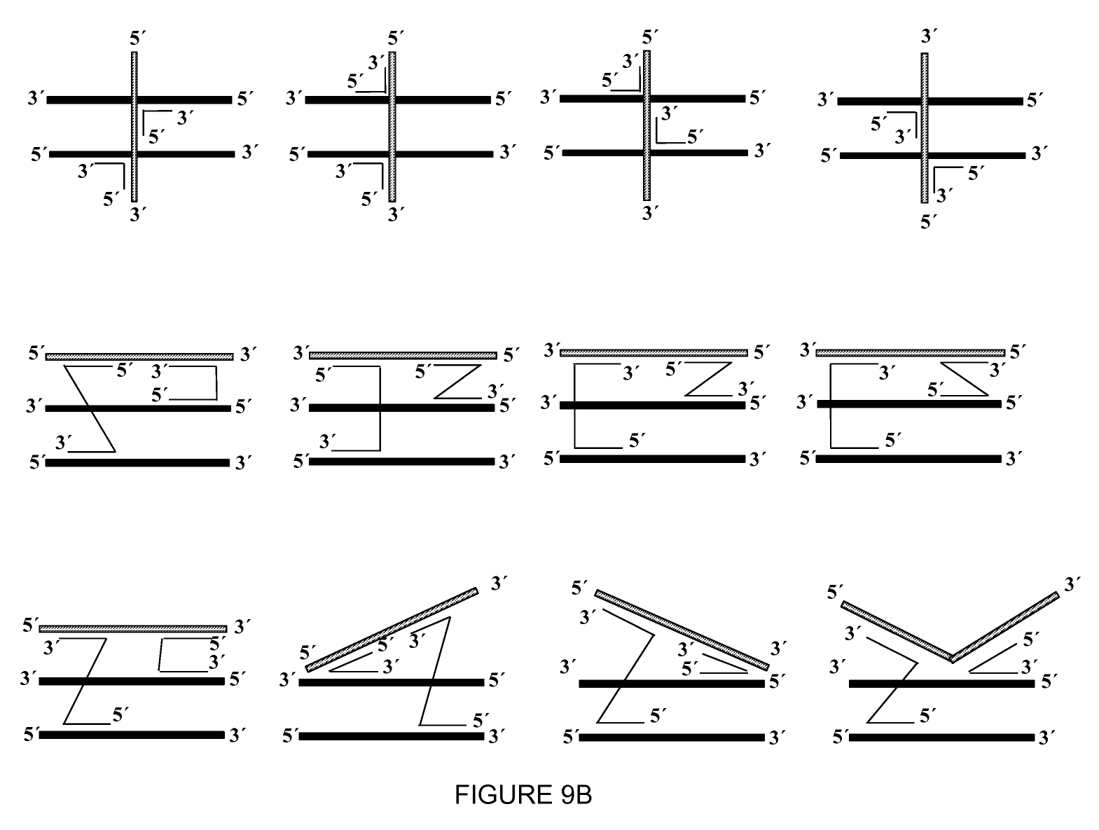

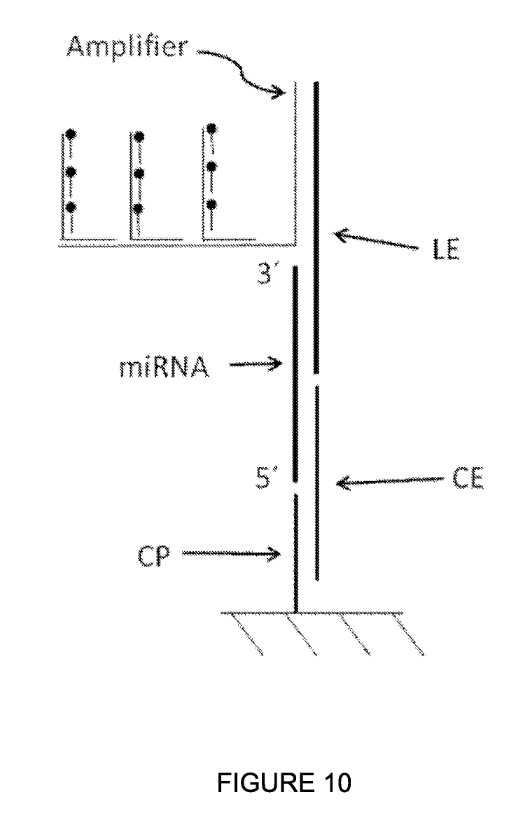

It is further noted that the label extenders may be in any of many different conformations. That is, the label extenders may be designed in the double-z (ZZ) configuration, the cruciform configuration, or any other related conformation as depicted, for instance, in FIGS. 9A and 9B. Each of these interchangeable conformations may be designed and utilized in these assays to achieve similar results. The structural variations of label extender probe design depicted in FIGS. 9A and 9B are only non-limiting examples and the Figures are do not depict all possible geometries or strategies. One of skill will immediately recognize that other useful and suitable label extender probe designs may be derived from these exemplary structures. More specifically it has been determined that especially the ZZ and the cruciform conformations work well in these assays. Furthermore, it is noted that various geometric alignments may be utilized in designing the cruciform and ZZ conformations, such as depicted in FIGS. 9A, 9B, 11A and 11B. FIGS. 11A and 11B are not intended to depict every possible design of the label extenders. Rather, these Figures merely depict specific embodiments of label extender design. One of skill in the art would be able to design other variations based on these themes which may also be suitable for the hereindescribed methodological embodiments.

Many different types of assays may be successful utilizing this multi-faceted approach to capture and detection. For instance, as will be explained in more detail below, this assay may be particularly useful for detecting single nucleotide polymorphisms (SNPs). Furthermore, because of the increased specificity and stability of probes comprising the cEt analogs, this assay method may be utilized to detect and quantitate micro-RNA (miRNA) species. Micro-RNA species are particularly difficult to detect due to their short sequence length, which is typically from approximately 11 to 22 nucleotides. This assay approach may be utilized to detect mRNA, DNA, siRNA, miRNA (mature and immature sequences), to detect SNPs, and utilized on, for instance, WGA samples, or any type of sample desired.

Generally, to perform SNP detection in vitro or in situ using this method, it is useful to design the label extender probes such that the SNP resides in the middle of the L-1 sequence, which may be comprised entirely of cEt molecules. Tissue samples or individual cells or groups of cells may be utilized directly and incubated with the components of the amplification architecture (probes and labels, described herein, etc.). or sample DNA may be removed from tissue and cells and sheared prior to performing the assay such that the average length is 20-50 nucleotides, or 30-50 nucleotides, or 40-50 nucleotides, using methods known in the art. This method may also be used to detect nucleic acid targets in intact cells which are non-adherent, i.e. circulating cells. At least approximately 50,000 cells should be harvested per sample in embodiments wherein the nucleic acid targets are first isolated from the cells/tissues. However, this is merely a general guide and as many as 100,000 cells, or more may be used to increase the robustness of the assay. Depending on the cell type, anywhere from 50,000 to 250,000 cells may be harvested and prepared for each sample. Alternatively, at least approximately 200-400 ng of nucleic acid material (RNA or DNA depending on the assay type) may be prepared for each sample. The amount of nucleic acid material or genetic material required for optimum results will vary depending on target sequence identity and probe design, but may be at least 150 ng, 200 ng, 250 ng, 300 ng, 350 ng, 400 ng, or 500 ng for each sample.

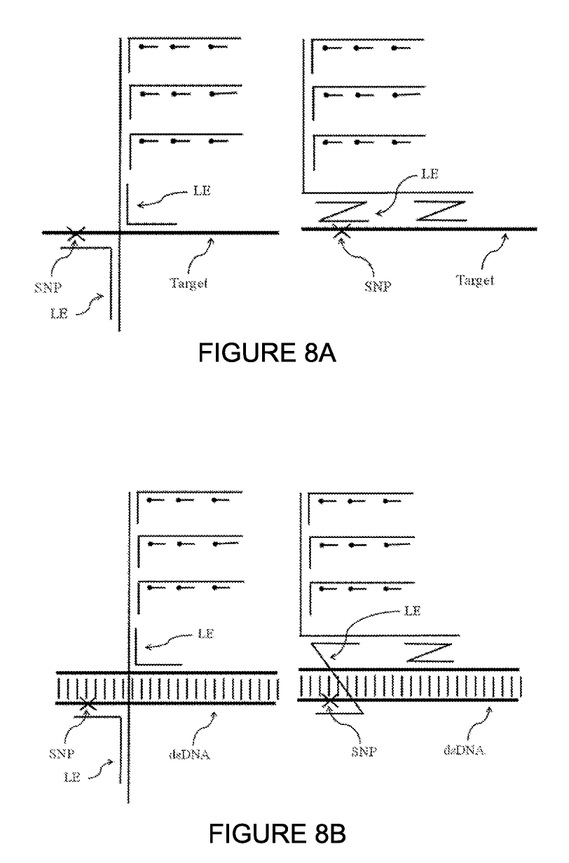

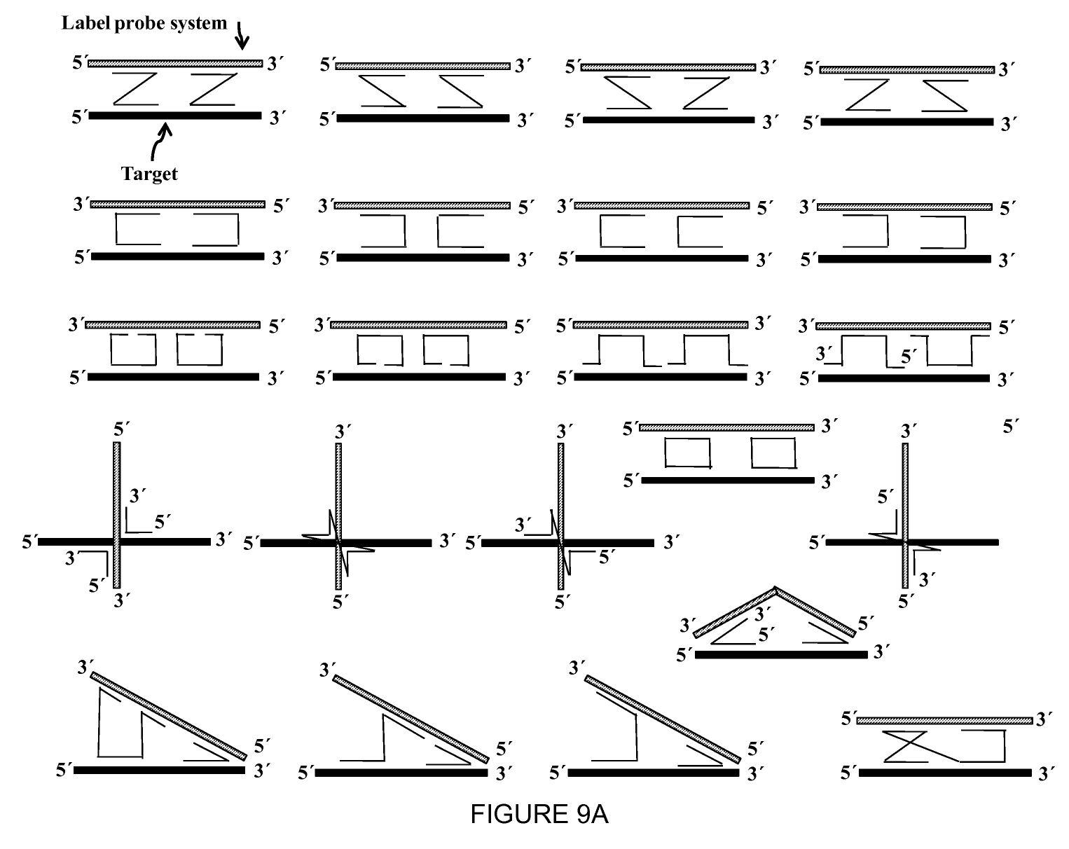

The above embodiments may also be implemented in such a way so that the L-1 sequence is able to distinguish between various SNP identities at a specific position within the chromosome, genome, genetic material, message RNA, miRNA, DNA (both single stranded and double stranded), or other sample to be tested. That is, the L-1 sequence may be designed such that the SNP which is to be detected resides in the very middle of one of the L-1 sequences on one of the label extender probes, or another location which provides optimal sensitivity. It should be noted that the label extender probes may be designed such that the L-1 sequence complementary to the SNP is in either one of the two LE probes. The use of nucleic acid analogs, such as LNA, PNA, cEt, etc. enables label extenders to bind to only the SNP allele that is perfectly complementary to the L-1 sequence. If the label extender binds to target, and subsequently the other components of the labeling system are added and detected, then the sequence of the SNP in the target nucleic acid may be determined. The label extender can further be designed such that it will only bind to a perfectly complementary target sequence such that if the identity of the nucleic acid at the SNP position is not perfectly complementary to the corresponding L-1 position, the label extender will not bind and no signal will be detected. A general depiction of one embodiment of this assay is provided in FIGS. 8A and 8B. Though FIGS. 8A and 8B illustrate the use of label extender probes both in the cruciform (left panels) and double Z (right panels) configurations for both single stranded targets and double stranded targets, other label extender configurations, such as those depicted in FIGS. 9A and 9B, may be equally effective in this assay. For instance, one could also employ the cruciform label extender geometry to achieve the same results.

As previously mentioned, the length of label extender probes in the present SNP detection embodiment may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Further, the desired melting temperature may also be target-dependant. That is, if a specific miRNA or SNP target is rich in, or has a high content of, G and C bases, then perhaps less melting temperature-increasing nucleic acid analogs, like cEt, will be necessary to achieve the desired melting temperature, as compared to a target region which is rich in A and T bases. In summary, design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

Optionally, the assay may be designed such that one specific label is utilized for detection of one allele of the SNP, and a second distinguishable label is used for detection of another allele of the SNP. In this optional embodiment, a signal will always be detected regardless of the identity of the SNP allele and the person conducting the experiment need only to distinguish between detection of both types of signals. In this way a sample can be identified as being homozygous for either allele, if a single label is detected, or heterozygous if signal for both labels is detected.

In another embodiment, the label extender probe may be a semicircle which has a break at the location of the SNP. Genotyping may then be achieved by ligation of the label extender loop by replacing the single missing base with, for instance, a labeled base. Alternatively, the label extender probes may simply be fully circular DNA probes wherein the L-1 sequence and the L-2 sequence are located at opposite ends of the circle, as depicted in FIG. 9A. It should be apparent to one of skill in the art that any of the label probe extender designs depicted in FIG. 9A may be swapped with any other label extender probe design, such that, for instance, perhaps one label probe is of the full circle variety while another may be of the “Z” variety, and the like. This “mix and match” property of the label probes is applicable to all of the embodiments provided herein, not just the present embodiment.

In another embodiment, the present assay is utilized to genotype multiple SNPs in a given stretch of target nucleic of interest. For instance, there may be multiple, linked SNPs in a specific region of the genome of interest and to be assayed. It is often desirable to determine whether SNPs localized in a small genomic region are “linked.” That is, it is of interest to determine if one or more SNP's in a specific region of the genome, localized in a short sequence, are identified as either one allele or the other, at the same time. The term “genetic linkage” described the tendency of certain loci or alleles to be inherited together. Genetic loci on the same chromosome are physically close to one another and tend to stay together during meiosis, and thus are considered in the art to be “genetically linked.” From SNP genotyping, linkage maps may be generated showing the relationship between various SNPs in a specific region of the genome, and it may be determined whether these SNPs are linked. The present assays allow multiple SNP genotyping by simply associating specific labels for each SNP location and allele sequence. Thus, one SNP location may have, for instance, two alleles, one which is labeled with a “green” fluorescent label through its specifically assigned label probe system, and one which is labeled with a “yellow” fluor in its specifically assigned label probe system. A second SNP location just downstream or upstream of the first SNP may be similarly genotyped using the same colors or two distinguishably different colors. If the two SNP locations are close enough, label extender probes may be designed such that each of the label extender probes in a pair of label extender probes may have an L-1 sequence perfectly complementary to one possible allele each of the closely located SNPs. Separate label probe extenders comprising different L-1 sequences which are perfectly complementary to the other alleles may also be used, and each specific pair of label probe extenders then designed to hybridize to their own specific label probe system, thereby genotyping multiple SNPs in a single assay.

In another related embodiment to the above, to control for false positives which may arise in the assay, the degree of certainty of the capture of a specific target nucleic acid and a specific SNP encoded therein may be made more robust by including a second target just downstream or upstream of the SNP target. That is, a second target sequence, not known to encode a SNP, lying just upstream or just downstream of the first target sequence containing the SNP may be also targeted in a multiplex assay. Furthermore, the pre-amplifier and amplifier and label probe system associated with the second target sequence could be labeled with a different label than the first target sequence containing the SNP. Thus, a true positive will only result when both the first target nucleic acid and second target nucleic acids are detectable and both different kinds of labels are detected. The second target sequence acts as a “positive control” to be sure the assay is functioning properly in the near vicinity of the target SNP. If the second target sequence is not captured and not labeled, this “positive control” lying just upstream or downstream of the target SNP nucleic acid sequence will be the indication that the assay is not functioning properly. For example, if the first target sequence is labeled with a fluorescent dye providing a “red” color to the detector, and the second target sequence is labeled with a fluorescent dye providing a different wavelength color or a shifted color, indication that both or present in the same vicinity of the sample means that the assay is working properly and the positive signal for the first target sequence is more likely than not a true positive. This embodiment may be varied in many ways, including use of any of the labels disclosed herein or known in the art which can be distinguished from each other. The target may be single stranded or double stranded nucleic acid. The target may not even comprise a SNP but merely comprise some other genetic abnormality which the use desires to know whether the two target sequences are located in close proximity to each other either with respect to sequence location or with respect to tissue type or cell type or chromosome, etc. The target nucleic acid, as explained elsewhere throughout this disclosure, may be DNA, RNA, and any and all species of such, such as, for instance miRNA (pre-, pri-, etc.). The secondary target sequence may be located any number of nucleic acids nearby the first target sequence. For instance, the secondary target sequence (or “positive control”) may be located 20 bp, 30 bp, 40 bp, 50 bp, 60 bp, 70 bp, 80 bp, 90 bp, 100 bp, 200 bp, 300 bp, 400 bp, 500 bp, 1 kb, 1.5 kb, 2 kb, 3 kb or even 4 kb upstream or downstream of the first and/or primary target sequence.

In other embodiments of this type, as already mentioned above, double stranded DNA may be detected using these compositions and methods. To make the detection of double stranded target detection more robust, it may be desirable to adjust the conditions and sequences of the label extenders which bind the dsDNA targets such that one LE binds its target more weakly than the LE binding the complimentary strand of the dsDNA target. It may further be desirable in some situations and under some conditions for some assays to design the probes such that the non-allele-specific LE binds the dsDNA target more weakly than the allele-specific LE, especially when assaying, for instance, SNPs and such. Further, one LE, such as the allele-specific LE, may contain LNA or other DNA analogues (such as cEt) whereas the other LE, such as the non-allele-specific LE, may be devoid of any such DNA analogues.

Yet another embodiment for increasing the robustness of the above methods, assays, systems and compositions, is to first pre-incubate the sample with pre-amplifier and/or amplifier. This incubation may be performed any amount of time prior to conducting the above-disclosed assays or methods. That is, the pre-incubation may be performed 1 m, 2 m, 3 m, 4 m, 5 m, 6 m, 7 m, 8 m, 9 m, 10 m, 20 m, 30 m, 1 hr, 1.5 hr, 2 hr or even 4 hr prior to adding the LE's and the remainder of the assay system components (where “m” is minutes). The pre-amplifier and/or amplifier sequences used in the pre-incubation step may be incubated within this step for any amount of time sufficient in which to allow the sequences to bind to the sample nucleic acids depending on the conditions, such as, for instance, 1 m, 2 m, 3 m, 4 m, 5 m, 6 m, 7 m, 8 m, 9 m, 10 m, 20 m, 30 m, 1 hr, 1.5 hr, 2 hr or even 4 hr.

### B. Micro-RNA Detection

As described above, AFFYMETRIX® provides compositions and products referred to as the QUANTIGENE® line of products. These kits and compositions usually utilize anywhere from between six and twelve capture extender probes and twelve to twenty four label extender probes. However, kits do not necessary need to be bound by these number of probes per kit and may comprise any number of probes, as desired or as suitable for detection of the desired number of targets. The melting temperature selected for these assays is typically from 62° C. to 67° C. The average length of the probes is about 24 nucleic acids. However, mature miRNA is typically in the range of 15-28 nucleic acids in length and have an average melting temperature of around 64° C. Thus, design of probesets useful in the QUANTIGENE® model for detection and quantitation of mature miRNA is difficult. To address this problem, the present embodiment incorporates use of LNA or other nucleic acid analogs to allow adjustment of hybridization regions L-1 and probe lengths to enable detection of mature miRNA. This present embodiment is designed such that the probes will hybridize only if the miRNA is in the mature form. (See, for instance, FIG. 10, representing an embodiment of capturing and detecting mature miRNA, this would not work if the miRNA were in pre-mature form since the hairpin-loop structure and excess sequences surrounding the mature miRNA would interfere with binding of the probes). Any miRNA species that are longer than the mature miRNA, i.e. such as with pre-miRNA, will not be detected. Further, the assay employs LNA, such as, for instance, cEt and analogs thereof, in the L-1 sequence which also allows for detection of the presence of mutations in the complementary regions of the target miRNA. In other words, use of cEt and like analogs can allow for sequencing of miRNA.

Referring to FIG. 10, depicted is one embodiment of the miRNA assay design. Attached to a support is a capture probe. A capture extender is provided which has a region C-1 which is complementary to the capture probe. The capture extender also comprises a sequence C-3 which is complementary to a sequence in the miRNA and is typically comprised of one or more nucleic acid analogs to increase specificity and stability of the resultant hybridization pair created by hybridization of the capture probe to the miRNA target sequence. One or more label extender probes are also provided comprising the L-1 and L-2 sequences as above, which may also be comprised of one or more nucleic acid analogs which provide the same melting temperature properties as previously described. The remainder of the typical label probe system then is allowed to hybridize to the label extender probe providing detectable signal.

The sequence C-3 may be generally as short as 7 nucleotides, 12 nucleotides, 11 nucleotides or even 9 nucleotides or fewer and still function adequately to capture the target miRNA due to the use of highly selective complementary nucleic acid analogs in the C-3 sequence. Likewise, the sequence L-1 may be generally as short as 7 nucleotides, 12 nucleotides, 11 nucleotides or even nucleotides or fewer and still function adequately to capture the target miRNA due to the use of highly selective complementary nucleic acid analogs. The quantity of nucleic acid analog present in this embodiment, as in other embodiments, varied depending on the required sensitivity. The sequences L-1 and C-3 may be 100% nucleic acid analog, or less, such as 90%, 80%, 70%, 60%, 50%, 40%, 30%, 20% or even as low as 10% nucleic acid analog content. By adjusting the content of nucleic acid analog in sequences C-3 and L-1, the assay may detect even very closely related miRNA homologs. Further, the L-1 sequence may be as long as 50% of the entire miRNA or siRNA sequence. Likewise the C-3 sequence may be as long as 50% of the entire miRNA or siRNA sequence, such that when added together, the L-1 sequence and C-3 sequence together are the same length as the miRNA or siRNA sequence when added together, such that the entire miRNA or siRNA sequence is completely hybridized to these two probes. Alternatively, one or more bases of the target siRNA or miRNA sequence may be unhybridized to any other probe during the assay, i.e. not a target of, or in any way hybridized to, the capture or label probe system components. In general the number of target nucleic acid bases not hybridized to one or more components of the assay components would be 0, 1, 2, 3, 4, 5 or as many as 10 nucleic acids.

The length of label extender probes may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Further, the desired melting temperature may also be target-dependant. That is, if a specific miRNA, expression target, translocation event sequence or SNP target is rich in, or has a high content of, G and C bases, then perhaps less melting temperature-increasing nucleic acid analogs, like cEt, will be necessary to achieve the desired melting temperature, as compared to a target region which is rich in A and T bases. In summary, design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

Various embodiments of this assay may be employed both in vitro and in situ, as needed. That is, the assay may be redesigned without the capture extender probes such that the detection occurs entirely in situ without the aid of a solid support. Again, as in other embodiments, the label extenders may be designed in various configurations as depicted in FIGS. 9A and 9B, i.e. such as, for example, a ZZ configuration or a cruciform configuration. It will also be clear to one in the art that similar methodologies can be applied to any nucleic acid target of this length or shorter, such as siRNA, for instance.

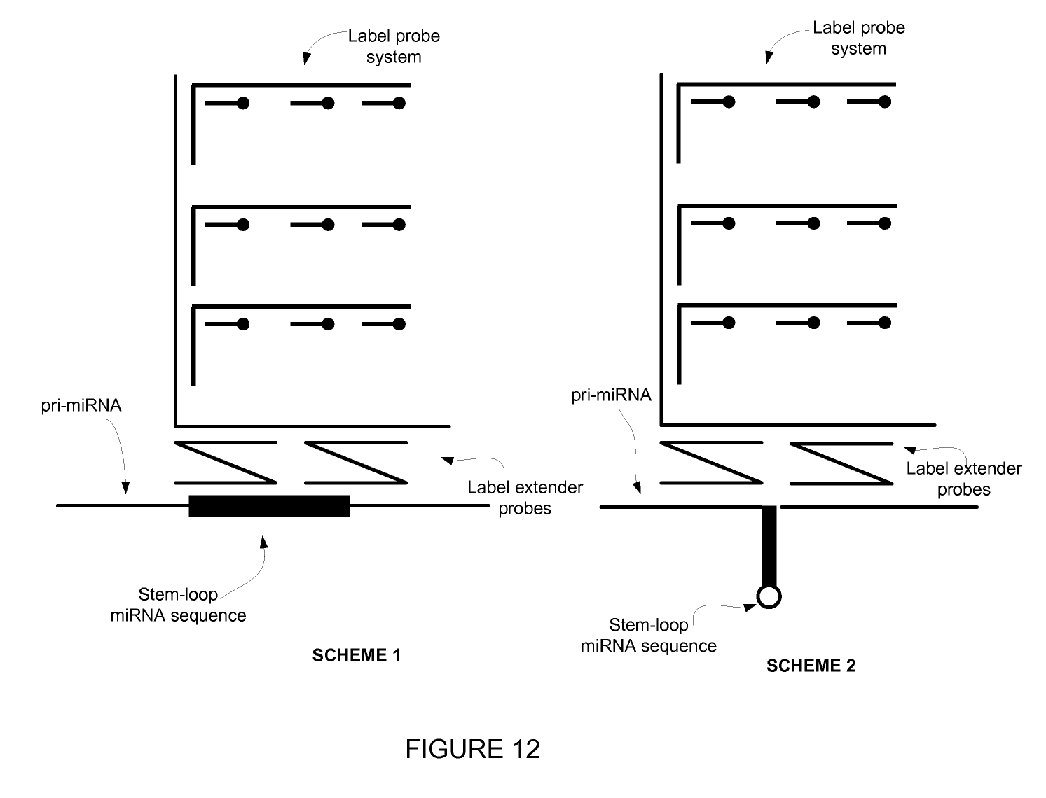

In another embodiment, mature miRNA may be distinguished from immature miRNA as depicted in FIG. 12 Immature miRNA is commonly referred to as pri-miRNA, where the stem-loop structure has not yet been acted upon by the RNase enzyme, for instance Drosha and/or Pasha. Thus, this embodiment allows detection of pri-miRNA prior to cleavage to remove the extraneous sequence extending beyond the common stem-loop structure of miRNA and prior to being acted upon by enzymes such as Dicer. In FIG. 12, there is shown three schemes which may be used to design LEs such that the LEs only bind to immature miRNA, i.e. miRNA that has not yet been converted by excision of the intervening stem/loop structure to fully mature miRNA. This embodiment may be used by itself in an assay or in conjunction with the assays, methods and compositions described above and depicted in FIG. 10. The present embodiment may be used without any support or microparticle for the capture step (this is optional), thus allowing for detection of immature miRNA in vitro, in situ and/or in vivo. In FIG. 12, the black box represents the miRNA sequence and the straight lines extending beyond the black box are the single stranded RNA extraneous regions present only in pri-miRNA. As shown in Scheme 1 of FIG. 13, the presence of pri-miRNA may be detected when the LE is designed such that the L-1 sequence complimentary to the target overlaps the extraneous regions upstream and downstream of the regions of miRNA which encode the stem-loop structure. Thus, only when the extraneous portions of the pri-miRNA sequence are present will the label probe system hybridize to the target and elicit a signal indicating the presence of the pri-miRNA sequence. Scheme 2 of FIG. 12 depicts an alternative embodiment of the same approach.

### C. Multi-Color FISH Embodiments and Detection of Gene Fusion/Translocation Events

The above-described methods and compositions may also be applied in embodiments in which the sample comprises purified chromosomes, as previously mentioned. Prior to the development of FISH techniques, scientists typically used what was termed “G-banding” to identify gross chromosomal abnormalities. G-banding allows detection of abnormalities of the chromosome based on size, number, centromere position and banding pattern, i.e. karyotyping of whole chromosomes using a light microscrope. With the advent of molecular genetics, these techniques were improved upon, culminating in the present day FISH techniques which accomplish the same goals, and more, and with much higher accuracy. FISH uses fluorescently-labeled DNA probes to hybridize to specific genetic regions of the chromosome which correlate with known linkages to diseases and genetic abnormalities. FISH utilizes fluorescent microscopy to aide in the detection of these regions. FISH is often used in laboratories testing samples for genetic abnormalities in conjunction with microarray technologies, which provides for independent validation of detection and diagnoses. FISH techniques have been in use in the scientific and clinical fields for at least the last 20 years.

Karyotyping using the old G-banding techniques typically provided 3 to 5 Mb resolution, i.e. was able to detect abnormalities that result in a change in the chromosome of 3 to 5 Mb, which only allows for detection of gross abnormalities. FISH, on the other hand, is still used today and can provide resolution of about 60 to 100,000 base pairs using fluorescence microscopy and labeled DNA probes. This type of resolution enables detection of translocations, may be combined with phenotypic analyses, and allows for visualization of rearrangements and aberrations and resultant positions of involved genomic sequences. Other FISH techniques utilize fosmids which are 40 kb in length. Bac clone resolutions achievable utilizing fosmid approaches using Bac clones are on the order of about 40 kb to 300 kb. FISH analysis may be utilized in the areas of clinical research, medical genetics, prenatal testing, pharmacogenomics (for example in the cancer treatment field), carcinogenesis, and postnatal testing. More broadly, the field of use of the present methods may be considered the molecular cytogenetics market. Most cytogenetics testing laboratories continue to use FISH techniques to validate and provide independent diagnoses.

The various abnormalities that may be detected by FISH include SNP detection, CNV detection, translocations and other such related abnormalities, i.e. LCH, LOH, UPD, etc. The various disease which may be associated with such genetic abnormalities may include, but are not limited to, for instance, cancer, developmental delay, autism, central nervous system disorders, heart diseases such as coronary artery disease, diabetes, psychological disorders such as schizophrenia, etc.

The methods disclosed herein may be applied within the FISH technology field to offer enhanced and broadened applications. For instance, use of nucleic acid analogs, such as but not limited to, cEt molecules, locked nucleic acids, PNA's, and other such analogs, will provide increased sensitivity and stability of prepared samples, while use of multicolor fluorescent tags which specifically hybridize to specific label extender probes offer the capability to perform multiplex FISH analysis, identifying multiple targets in a single sample and in a single test.

More specifically, the label extenders may be any number of possible and suitable conformations, such as the ZZ or cruciform, or any other suitable conformation as illustrated in the non-exhaustive examples depicted in FIGS. 9A and 9B. The sequence L-1 in the label extender in these embodiments is partially or completely comprised of nucleic acid analog molecules to provide the increased melting temperature and the desired amount of specificity and stability for the label probe machinery interaction with the target sequence. There is no need in this application for a capture probe or capture extender since the label extender binds directly to the chromosomal material.

It is further noted that in this embodiment, and others, simultaneous detection of both RNA and DNA targets may be achieved. Further, RNA and DNA targets may be distinguished from each other in the same assay. For instance, considering the structures of label extender probes depicted in FIG. 9B, one could design one set of label extender probes which are specific for double-stranded DNA as in this Figure, and design label extender probes specific for single-stranded RNA species as in FIG. 9A, which may be utilized in a single assay. Providing amplifiers and pre-amplifiers with specific recognition sequences distinct for two different label probe systems, one zip code address for the double-stranded label extender probe set and a different zip code address for the single-stranded label extender probe set, allows for discrimination and detection of both RNA and DNA species in a single assay. This design approach may be utilized in the present embodiment, as well as in embodiments using encoded microparticles, other in situ or in cellulo embodiments, miRNA detection embodiments, SNP detection embodiments, and fusion/translocation embodiments as described in further detail below.

Samples may be prepared and processed on a slide according to any of many well known procedures provided either on the Panomics website or other websites. (See, for instance, Nature Methods, 2:237-238, 2005 and references cited therein). The internet offers a plethora of protocols for FISH sample preparation. Samples may be, for instance, cells grown in culture, isolated cells from primary sources, tissue slices, purified chromosomes as in traditional karyotyping, and other samples suitable for FISH analysis. Once prepared either on a slide or in a well, the hybridization step may be accomplished by any of the methods also provided in the literature. For instance, Panomics produces the QUANTIGENE® ViewRNA product and kits which are provided with a user's manual describing the protocol and methods in which samples may be prepared and hybridized, and which are known to one of skill in the art (see, www.panomics.com/index.php?id=product_{—}87#product_works_{—}87). The manual for this protocol, “QUANTIGENE® ViewRNA User Manual,” incorporated by reference in its entirety for all purposes, may also be downloaded from the Panomics website (see, panomics.com/downloads/UM15646_QGViewRNA_RevA_{—}080526.pdf, contents of which are incorporated herein by reference in its entirety for all purposes).

The length of label extender probes may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Further, the desired melting temperature may also be target-dependant. That is, if a specific miRNA or SNP target is rich in, or has a high content of, G and C bases, then perhaps less melting temperature-increasing nucleic acid analogs, like cEt, will be necessary to achieve the desired melting temperature, as compared to a target region which is rich in A and T bases. In summary, design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

Once the label extender is hybridized to the target, the other components of the label machinery may be added to the sample to assemble the full labeling structure onto the sample. Alternatively, a labeling structure, which includes some or all of the labeling components depicted, for instance, in FIGS. 5A and 5B, may be partly assembled onto the label extender prior to hybridizing the label extender to the target sequence. Further, perhaps only the pre-amplifier and amplifier will be hybridized with the label extender probes prior to contact with the sample. In short, the various structure components depicted in FIGS. 5A and 5B, may be assembled in any order desired to achieve optimum signal and sample handling results. For instance, to minimize sample preparation steps, it may be advantageous to have all labeling structure components in a single tube which is added to the sample in a single step. However, if the resultant signal is not optimal, it may be necessary to add the various components of the label scaffolding in multiple steps or even one step at a time so each component hybridizes separately to its intended target component. Washing steps may be implemented between some or all of the steps of the process to optimize the desired result.

It should be noted that multiple fluorescent labels may be used in the present methods. That is, one of skill in the art is aware of the various labels in existence in the market place which may be amenable to the presently disclosed method. Further, it should be noted that various fluors or known to provide different ranges of fluorescent wavelength. Thus, one could build a labeling structure such that one target genomic sequence is hybridized with one specific set of labeling structures providing a specific fluor color or wavelength, whereas a second and different target sequence may be designed using the labeling machinery to bind a differently labeled set of fluors yielding a different fluor color or wavelength, allowing for multiplex assays with the FISH protocol.

In another embodiment, using similar components, an assay may be designed to detect the translocation of genes or fusion of genes, which is known to occur in various cancer diseases. For instance, the Philadelphia chromosome or translocation is associated with chronic myelogenous leukemia (CML) and is the result of a reciprocal translocation between chromosomes 9 and 22, designated t(9;22)(q34;q11). The resultant fused gene is the gene product BCR-ABL. Furthermore, it has been reported that the TMPRSS2-ETS gene fusion is associated with prostate cancer and is found in 50%-70% of prostate-specific-antigen (PSA)-screened hospital-based prostate cancers. (See, Kirsten et al., Other examples found in the literature includes the SLC45A3-ELK4 fusion. Presently FISH-based assays are used to detect these genetic events. However, FISH-based assays are only effective for DNA-based fusion events, not RNA-based fusion events such as the SLC45A3-ELK4 fusion.

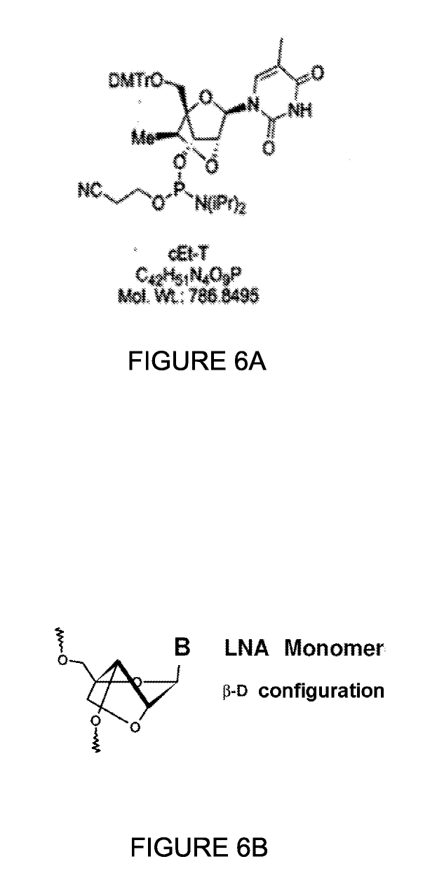

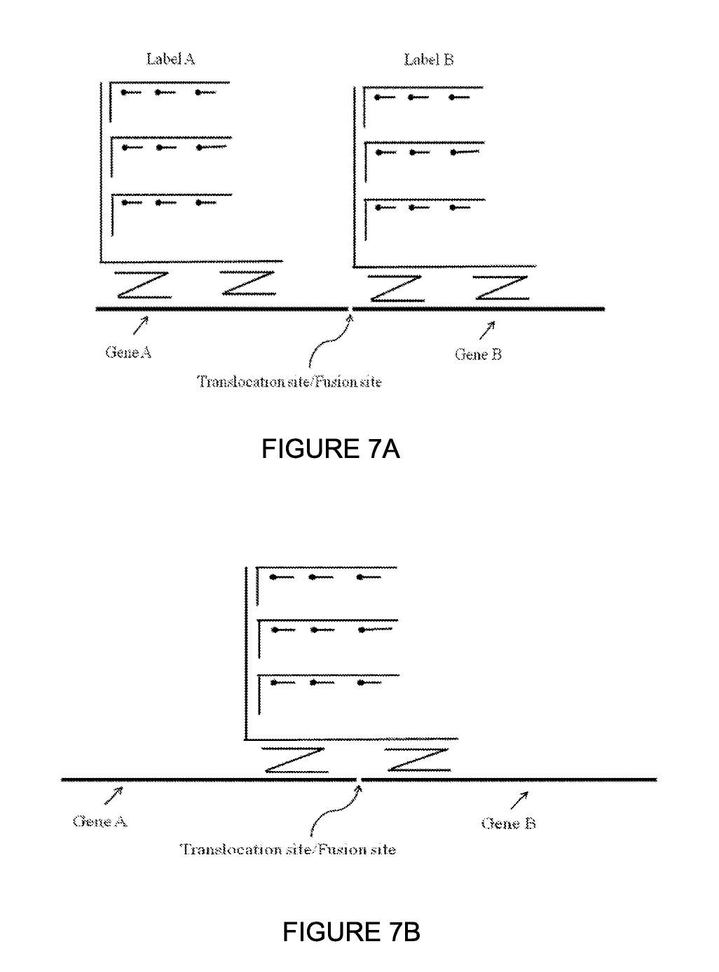

In a typical assay, a target sequence of between 500 to 1000 nucleic acids in length is sufficient to provide a sensitive assay signal. Optionally, the target sequence may be 300, 400, 500, 600, 700, 800 or even 900 to 1000 nucleic acids in length. In one embodiment of the assay, as depicted in FIG. 7A, separate pairs of label extenders are hybridized through their L-1 sequences to the two targets, i.e. one target being one-half of the fusion gene (such as, for instance, BCR) and the second target being the second half of the fusion gene (such as, for instance, ABL). The two sets of label extenders may be specific for specific pre-amplifiers hybridizing through the L-2 sequence such that each target is separately labeled with different labels, such as differently colored dyes. When viewed through a fluorescence microscope, for instance—if this is the detection method specific for the label utilized in the assay, then one would look for the co-localization of the two different labels. If the labels are not co-localized, then no translocation is detected. If the labels are co-localized, this is evidence of a translocation event of the type being assayed. Various labels may be employed for the two sets of label extenders such that when the two different labels are within proximity of each other, a FRET reaction may occur to generate a third color distinguishable from the first two colors. Thus, a translocation event positive signal would be indicated by the appearance of a third color. For instance, one set of label extender probes may hybridize to one specific label probe system with green fluorescent labels. A second label probe system may be employed with a different label probe system comprising, for example, a red fluorescent label. If there is a translocation event in the sample, these two different colored label systems will be found in very close proximity to each other and the combination of labels will interact to general a third distinctive color which may be detected and indicative therefore of the translocation event.

In a multiplex variation of the above, multiple colors may be utilized in the assay to detect multiple different genetic segments. Any number of different labels and thus different colors may be utilized, depending on what labels are available in the art. For instance, as many as 2, 3, 4, 5, 6, 7, 8, 9, 10, 12, 14, 16, 18, 20, 25, 30, 35 or even 40 detectably distinguishable labels may used in a single assay, each directed to a different gene sequence and therefore, each corresponding to a separate label probe system set of probes. Generally, but not necessarily, as described above, for each target, there would be included in the method, composition and system of the present invention, a different set of probes and different label probe system. Furthermore, these multiple labels each possessing multiple detectably distinguishable colors, could be used for determination of copy number of specific target nucleic acids, genes, genetic sequences, etc. For instance, it is known, as also discussed above, that various diseases are associated with copy number abnormalities. Thus, it is of interest to be able to detect the number of copies of specific target sequences. This may be accomplished by assigning a different label for each different copy of the sequence. Thus, the more labels that bind, each detectably distinguishable from the other, the more copies, or fewer copies as the case may be, of the target sequence that is present in the sample.

It is noted that in these multicolor embodiments, the multiple detectably distinguishable labels may be arranged in different ways in the label probe system. For instance, it may be possible to include multiple different labels all attached to a single amplifier, which is in turn attached to a pre-amplifier. Thus, the single amplifier will have multiple different labels. In another embodiment, the first single amplifier could comprise a single label type, and a second single amplifier next to it, also hybridized to the same pre-amplifier, could have hybridized thereto label probes comprising a detectably different label from the first single amplifier. Thus, a pre-amplifier may have hybridized thereto a first and a second amplifier, wherein the first amplifier comprises hybridized thereto a first label probe type and the second amplifier comprises hybridized thereto a second label probe type wherein the first label probe type is detectably distinguishable from the second label probe type. Thus, if there are, for instance, only four or six different label probe types, these different label probe types may be combined and mixed and matched onto various different amplifiers to create combinations which would then extend the multiplexing ability of the assay based on these labels.

For instance, if the goal is to detect metaphase chromosomes using the present methods and compositions, one must be very careful to mitigate false positives. This embodiment may applied equally as well to detection of interphase genetic material. To mitigate false positives, multiple adjacent locations on the chromosomes may be labeled with different colors to increase specificity. The user performing the assay then would only make a positive determination (“a call”) if both colors are present. Since the data will be punctate, one can tell when they are collocated. Since one expects a relatively low number of spots to light up in metaphase and they will be well separated, one can use all possible combinations of colors. Cytogeneticists typically image using a 60-100× objective for FISH. Assuming the assay comprises four different labels and corresponding label probe systems, i.e. red, yellow, green, and blue, it is possible to distinguish up to six different pairs of colors or punctuate dots. If the assay comprises six different labels and corresponding label probe systems, it may be possible to distinguish up to fifteen pairs of punctuate dots in a single assay. Thus, for “N” colors, it is possible to utilize in a single assay N*(N−1)/2 combinations. There is little reason not to use 2, 3, to 4 together, which provides 13 combinations from a set of four labels and 47 from a set of labels. Then N labels provides 2^{(N-N-1)} combinations of two or more labels. Beyond metaphase, this color expansion also may be applied to molecules which are well separated relative to the imaging and where the labeling is reasonably quantitative. In addition, in applications or situations in which the sample is known to not contain very low copy number of target nucleic acid sequence per cell, i.e. where the goal is to precisely quantitate 0 CN vs 1 CN vs 2 CN per cell, then the assay may utilize the singletons such that the number of combinations is then 2^{N-1}.

In another embodiment, as depicted in FIG. 7B, a single label extender may be made which traverses the translocation site. In this embodiment, detection of a signal indicates the genetic event being detected has occurred. No signal in this embodiment is indicative of no translocation/fusion genetic event in the test sample.

The length of label extender probes may vary in length anywhere from 10 to 60 nucleic acids or more, i.e. 11, 13, 15, 17, 19, 21, 25, 30, 35, 40, 45 or 50 nucleic acids in length. The sequence L-1 will also vary depending on the identity of the target and the number of potentially cross-reacting probes within the hybridization mixture. For instance, L-1 may be anywhere from 7 to 50 nucleic acids in length, or 10 to 40, or 12 to 30 or 15 to 20 nucleotides in length. The sequence L-1 may be entirely comprised of nucleic acid analogs or only partly comprised of nucleic acid analogs. For instance, it may be that every other nucleic acid is an analog in L-1, providing a 50% substitution of analog for native or wild type base. Alternatively, the L-1 sequence may be 100% comprised of nucleic acid analog. Further the L-1 sequence may be 10%, 20%, 30%, 40%, 50%, 60%, 70%, 80% or 90% comprised of nucleic acid analog. The underlying principle to the use of nucleotide analogs, such as cEt, is to increase the melting temperature or temperature at which the L-1 sequence remains hybridized to the target sequence. Typically, the LE and CE may be designed such that the target melting temperature for the assay is in the range of 50° C. to 56° C., or 49° C. to 57° C., or 48° C. to 48° C., etc. However, this may vary depending on buffer conditions and assay. For instance, when performing an in situ assay, it may be useful to add a neutralizing or denaturing agent such as formamide, and thereafter adjust the target melting temperature downwards to a range of 40° C. to 50° C. or lower. Thus the amount of melting temperature-increasing nucleotide analog present in L-1 can be doped up or down to the desired and empirically-determined most suitable amount to achieve the desired melting temperature, which will in turn provide the best performance with respect to affinity and specificity. Further, the desired melting temperature may also be target-dependant. That is, if a specific miRNA or SNP target is rich in, or has a high content of, G and C bases, then perhaps less melting temperature-increasing nucleic acid analogs, like cEt, will be necessary to achieve the desired melting temperature, as compared to a target region which is rich in A and T bases. In summary, design of the L-1 sequence, as in any probe sequence binding to the target, and determination of the amount of nucleotide analog to use in a specific embodiment of the presently disclosed assays, will depend on many factors including target sequence, buffer conditions and melting temperature needed to achieve the desired specificity and affinity in the assay.

An example of such as an assay is depicted in FIG. 7. Though FIGS. 7A and 8B depict the use of label extenders in the ZZ formation, other suitable label extender structures may be designed and utilized in the assay, such as the cruciform formation or other formations as depictured in FIG. 9. Furthermore, the two different sets of label extenders may utilize different conformations, i.e. one using ZZ conformation for one-half of the target, and the other label extenders for the other half of the target sequence being in a different conformation selected from those depicted, for instance, in FIG. 9. Additionally, in some assays, additional sets of label extenders may be employed, i.e. more than just two per target. For instance, an assay may be designed which utilized any number of label extenders in multiples of two. Thus, the assay may be designed such that half of the translocation target sequence is bound by as many as 2, 4, 6, 8, 10, 12, 14 or any multiple of two between 16-40 label extenders. The other half of the target may be designed to be bound by the same number, or a different number of label extenders.

### Compositions

Compositions related to the methods are another feature of the invention. Thus, one general class of embodiments provides a composition for detecting two or more nucleic acids of interest. In one aspect, the composition includes a pooled population of particles. The population comprises two or more subsets of particles, with a plurality of the particles in each subset being distinguishable from a plurality of the particles in every other subset. The particles in each subset have associated therewith a different capture probe. In another aspect, the composition includes a solid support comprising two or more capture probes, wherein each capture probe is provided at a selected position on the solid support.

The composition also optionally may include two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the nucleic acids of interest, and the capture extenders in each subset are capable of hybridizing to one of the capture probes and thereby associating each subset of n capture extenders with a selected subset of the particles or with a selected position on the solid support. Similarly, each subset of m label extenders is capable of hybridizing to one of the nucleic acids of interest.

The composition optionally includes a sample comprising or suspected of comprising at least one of the nucleic acids of interest, e.g., two or more, three or more, etc. nucleic acids. Optionally, the composition comprises one or more of the nucleic acids of interest or target nucleic acids. In one class of embodiments, each nucleic acid of interest present in the composition is hybridized to its corresponding subset of n capture extenders, and the corresponding subset of n capture extenders is hybridized to its corresponding capture probe. Each nucleic acid of interest is thus associated with an identifiable subset of the particles. In this class of embodiments, each nucleic acid of interest present in the composition is also hybridized to its corresponding subset of m label extenders. The component of the label probe system (e.g., the amplification multimer or preamplifier) is hybridized to the m label extenders. The composition is maintained at a hybridization temperature that is greater than a melting temperature Tₘ of a complex between each individual label extender and the component of the label probe system (e.g., the amplification multimer or preamplifier). The hybridization temperature is typically about 5° C. or more greater than the Tₘ, e.g., about 7° C. or more, about 10° C. or more, about 12° C. or more, about 15° C. or more, about 17° C. or more, or even about 20° C. or more greater than the Tₘ. Where in situ applications are called for, the capture probe, capture extenders and particles are not included in the compositions.

Essentially all of the features noted for the methods above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Another general class of embodiments provides a composition for detecting one or more nucleic acids of interest. The composition includes a solid support comprising one or more capture probes, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture probes and thereby associating each subset of n capture extenders with the solid support. Each subset of m label extenders is capable of hybridizing to one of the nucleic acids of interest. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

In one class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises a pooled population of particles. The population comprises two or more subsets of particles. A plurality of the particles in each subset are distinguishable from a plurality of the particles in every other subset, and the particles in each subset have associated therewith a different capture probe. The capture extenders in each subset are capable of hybridizing to one of the capture probes and thereby associating each subset of n capture extenders with a selected subset of the particles.

In another class of embodiments, the one or more nucleic acids of interest comprise two or more nucleic acids of interest, or target nucleic acids, the one or more subsets of n capture extenders comprise two or more subsets of n capture extenders, the one or more subsets of m label extenders comprise two or more subsets of m label extenders, and the solid support comprises two or more capture probes, wherein each capture probe is provided at a selected position on the solid support. The capture extenders in each subset are capable of hybridizing to one of the capture probes and thereby associating each subset of n capture extenders with a selected position on the solid support.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

For example, the label probe system can include an amplification multimer or preamplifier, which amplification multimer or preamplifier is capable of hybridizing to the at least two label extenders. The composition optionally includes one or more of the nucleic acids of interest, wherein each nucleic acid of interest is hybridized to its corresponding subset of m label extenders and to its corresponding subset of n capture extenders, which in turn is hybridized to its corresponding capture probe. The amplification multimer or preamplifier is hybridized to the m label extenders. The composition is maintained at a hybridization temperature that is greater than a melting temperature Tₘ of a complex between each individual label extender and the amplification multimer or preamplifier (e.g., about 5° C. or more, about 7° C. or more, about 10° C. or more, about 12° C. or more, about 15° C. or more, about 17° C. or more, or about 20° C. or more greater than the Tₘ).

Compositions are also understood to comprise label extenders and capture extenders having one or more nucleic acid analogs. That is, the sequences of L-1 and C-3, may contain anywhere from 1% to 100% nucleic acid analogs, such as, for instance, cEt, LNA, PNA and the like, and mixtures thereof. With regard to cEt, it is understood that other nucleic acid analogs of similar structure and having the same or similar properties, i.e. the ability to increase the melting temperature of a hybridization event between the capture extender and/or label extender sequence and the target sequence. Thus, minor alterations to the structure of the cEt, including, but not limited to, addition of other alkyl groups, alkylene groups, thiols, amines, carboxyls, etc. which have similar chemical properties suitable to the assays and methods provided above, are also included in these compositions. Compositions are further intended to include those compositions designed specifically for detection of target nucleic acids in situ, which would not require the use of, and therefore not include in the composition, capture probes, capture extenders and/or particles.

### Kits

Yet another general class of embodiments provides a kit for detecting two or more nucleic acids of interest. In one aspect, the kit includes a pooled population of particles. The population comprises two or more subsets of particles, with a plurality of the particles in each subset being distinguishable from a plurality of the particles in every other subset. The particles in each subset have associated therewith a different capture probe. In another aspect, the kit includes a solid support comprising two or more capture probes, wherein each capture probe is provided at a selected position on the solid support.

The kit also includes two or more subsets of n capture extenders, wherein n is at least two, two or more subsets of m label extenders, wherein m is at least two, and a label probe system comprising a label, wherein a component of the label probe system is capable of hybridizing simultaneously to at least two of the m label extenders in a subset. Each subset of n capture extenders is capable of hybridizing to one of the nucleic acids of interest, and the capture extenders in each subset are capable of hybridizing to one of the capture probes and thereby associating each subset of n capture extenders with a selected subset of the particles or with a selected position on the solid support. Similarly, each subset of m label extenders is capable of hybridizing to one of the nucleic acids of interest. The components of the kit are packaged in one or more containers. The kit optionally also includes instructions for using the kit to capture and detect the nucleic acids of interest, one or more buffered solutions (e.g., lysis buffer, diluent, hybridization buffer, and/or wash buffer), standards comprising one or more nucleic acids at known concentration, and/or the like.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

Another general class of embodiments provides a kit for detecting one or more nucleic acids of interest. The kit includes a solid support comprising one or more capture probes, one or more subsets of n capture extenders, wherein n is at least two, one or more subsets of m label extenders, wherein m is at least two, and a label probe system comprising a label. Each subset of n capture extenders is capable of hybridizing to one of the nucleic acids of interest, and the capture extenders in each subset are capable of hybridizing to one of the capture probes and thereby associating each subset of n capture extenders with the solid support. Each subset of m label extenders is capable of hybridizing to one of the nucleic acids of interest. A component of the label probe system (e.g., a preamplifier or amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2. The components of the kit are packaged in one or more containers. The kit optionally also includes instructions for using the kit to capture and detect the nucleic acids of interest, one or more buffered solutions (e.g., lysis buffer, diluent, hybridization buffer, and/or wash buffer), standards comprising one or more nucleic acids at known concentration, and/or the like.

Essentially all of the features noted for the embodiments above apply to these embodiments as well, as relevant; for example, with respect to composition of the label probe system; type of label; inclusion of blocking probes; configuration of the capture extenders, capture probes, label extenders, and/or blocking probes; number of nucleic acids of interest and of subsets of particles or selected positions on the solid support, capture extenders and label extenders; number of capture or label extenders per subset; type of particles; source of the sample and/or nucleic acids; and/or the like.

For example, in one class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises a pooled population of particles. The population comprises two or more subsets of particles. A plurality of the particles in each subset are distinguishable from a plurality of the particles in every other subset, and the particles in each subset have associated therewith a different capture probe. The capture extenders in each subset are capable of hybridizing to one of the capture probes and thereby associating each subset of n capture extenders with a selected subset of the particles.

In another class of embodiments, the one or more nucleic acids of interest comprise two or more nucleic acids of interest, the one or more subsets of n capture extenders comprise two or more subsets of n capture extenders, the one or more subsets of m label extenders comprise two or more subsets of m label extenders, and the solid support comprises two or more capture probes, wherein each capture probe is provided at a selected position on the solid support. The capture extenders in each subset are capable of hybridizing to one of the capture probes and thereby associating each subset of n capture extenders with a selected position on the solid support.

Kits are also understood to comprise label extenders and capture extenders having one or more nucleic acid analogs. That is, the sequences of L-1 and C-3, may contain anywhere from 1% to 100% nucleic acid analogs, such as, for instance, cEt, LNA, PNA and the like, and mixtures thereof. With regard to cEt, it is understood that other nucleic acid analogs of similar structure and having the same or similar properties, i.e. the ability to increase the melting temperature of a hybridization event between the capture extender and/or label extender sequence and the target sequence. Thus, minor alterations to the structure of the cEt, including, but not limited to, addition of other alkyl groups, alkylene groups, thiols, amines, carboxyls, etc. which have similar chemical properties suitable to the assays and methods provided above, are also included in these kits. Kits are further intended to include those compositions designed specifically for detection of target nucleic acids in situ, which would not require the use of, and therefore not include in the kit, capture probes, capture extenders and/or particles.

### Systems

In one aspect, the invention includes systems, e.g., systems used to practice the methods herein and/or comprising the compositions described herein. The system can include, e.g., a fluid and/or microsphere handling element, a fluid and/or microsphere containing element, a laser for exciting a fluorescent label and/or fluorescent microspheres, a detector for detecting light emissions from a chemiluminescent reaction or fluorescent emissions from a fluorescent label and/or fluorescent microspheres, and/or a robotic element that moves other components of the system from place to place as needed (e.g., a multiwell plate handling element). For example, in one class of embodiments, a composition of the invention is contained in a flow cytometer, a Luminex 100™ or HTS™ instrument, a microplate reader, a microarray reader, a luminometer, a colorimeter, fluorescence micropscope, substrates (such as slides, well plates, etc.) on which samples may be prepared for assay, or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element and/or laser). The computer can also receive data from other components of the system, e.g., from a detector, and can interpret the data, provide it to a user in a human readable format, or use that data to initiate further operations, in accordance with any programming by the user.

### Labels

A wide variety of labels are well known in the art and can be adapted to the practice of the present invention. For example, luminescent labels and light-scattering labels (e.g., colloidal gold particles) have been described. (See, e.g., Csaki et al. (2002) “Gold nanoparticles as novel label for DNA diagnostics,” Expert Rev. Mol. Diagn., 2:187-93).

As another example, a number of fluorescent labels are well known in the art, including but not limited to, hydrophobic fluorophores (e.g., phycoerythrin, rhodamine, Alexa Fluor 488 and fluorescein), green fluorescent protein (GFP) and variants thereof (e.g., cyan fluorescent protein and yellow fluorescent protein), and quantum dots. (See, e.g., The Handbook: A Guide to Fluorescent Probes and Labeling Technologies, Tenth Edition or Web Edition (2006) from Invitrogen (available on the internet at probes.invitrogen.com/handbook), for descriptions of fluorophores emitting at various different wavelengths (including tandem conjugates of fluorophores that can facilitate simultaneous excitation and detection of multiple labeled species). For use of quantum dots as labels for biomolecules, see e.g., Dubertret et al. (2002) Science, 298:1759; Nature Biotechnology (2003) 21:41-46; and Nature Biotechnology (2003) 21:47-51. Other various labels are known in the art, such as Alexa Fluor Dyes (Life Technologies, Inc., California, USA, available in a wide variety of wavelengths, see for instance, Panchuk, et al., J. Hist. Cyto., 47:1179-1188, 1999), biotin-based dyes, digoxigenin, AttoPhos (JBL Scientific, Inc., California, USA, available in a variety of wavelengths, see for instance, Cano et al., Biotechniques, 12(2):264-269, 1992), etc.

Labels can be introduced to molecules, e.g. polynucleotides, during synthesis or by postsynthetic reactions by techniques established in the art; for example, kits for fluorescently labeling polynucleotides with various fluorophores are available from Molecular Probes, Inc. ((www.) molecularprobes.com), and fluorophore-containing phosphoramidites for use in nucleic acid synthesis are commercially available. Similarly, signals from the labels (e.g., absorption by and/or fluorescent emission from a fluorescent label) can be detected by essentially any method known in the art. For example, multicolor detection, detection of FRET, fluorescence polarization, and the like, are well known in the art.

### Microspheres

Microspheres are preferred particles in certain embodiments described herein since they are generally stable, are widely available in a range of materials, surface chemistries and uniform sizes, and can be fluorescently dyed. Microspheres can be distinguished from each other by identifying characteristics such as their size (diameter) and/or their fluorescent emission spectra, for example. Furthermore, as explained in better detail above, the particles may be microspheres which may also be microparticles having a code therein.

Luminex Corporation ((www.) luminexcorp.com), for example, offers 100 sets of uniform diameter polystyrene microspheres. The microspheres of each set are internally labeled with a distinct ratio of two fluorophores. A flow cytometer or other suitable instrument can thus be used to classify each individual microsphere according to its predefined fluorescent emission ratio. Fluorescently-coded microsphere sets are also available from a number of other suppliers, including Radix Biosolutions ((www.) radixbiosolutions.com) and Upstate Biotechnology ((www.) upstatebiotech.com). Alternatively, BD Biosciences ((www.) bd.com) and Bangs Laboratories, Inc. ((www.) bangslabs.com) offer microsphere sets distinguishable by a combination of fluorescence and size. As another example, microspheres can be distinguished on the basis of size alone, but fewer sets of such microspheres can be multiplexed in an assay because aggregates of smaller microspheres can be difficult to distinguish from larger microspheres.

Microspheres with a variety of surface chemistries are commercially available, from the above suppliers and others (e.g., see additional suppliers listed in Kellar and Iannone (2002) “Multiplexed microsphere-based flow cytometric assays” Experimental Hematology 30:1227-1237 and Fitzgerald (2001) “Assays by the score” The Scientist 15[11]:25). For example, microspheres with carboxyl, hydrazide or maleimide groups are available and permit covalent coupling of molecules (e.g., polynucleotide capture probes with free amine, carboxyl, aldehyde, sulfhydryl or other reactive groups) to the microspheres. As another example, microspheres with surface avidin or streptavidin are available and can bind biotinylated capture probes; similarly, microspheres coated with biotin are available for binding capture probes conjugated to avidin or streptavidin. In addition, services that couple a capture reagent of the customer's choice to microspheres are commercially available, e.g., from Radix Biosolutions ((www.) radixbiosolutions.com).

Protocols for using such commercially available microspheres (e.g., methods of covalently coupling polynucleotides to carboxylated microspheres for use as capture probes, methods of blocking reactive sites on the microsphere surface that are not occupied by the polynucleotides, methods of binding biotinylated polynucleotides to avidin-functionalized microspheres, and the like) are typically supplied with the microspheres and are readily utilized and/or adapted by one of skill. In addition, coupling of reagents to microspheres is well described in the literature. For example, see Yang et al. (2001) “BADGE, Beads Array for the Detection of Gene Expression, a high-throughput diagnostic bioassay” Genome Res. 11:1888-98; Fulton et al. (1997) “Advanced multiplexed analysis with the FlowMetrix™ system” Clinical Chemistry 43:1749-1756; Jones et al. (2002) “Multiplex assay for detection of strain-specific antibodies against the two variable regions of the G protein of respiratory syncytial virus” 9:633-638; Camilla et al. (2001) “Flow cytometric microsphere-based immunoassay: Analysis of secreted cytokines in whole-blood samples from asthmatics” Clinical and Diagnostic Laboratory Immunology 8:776-784; Martins (2002) “Development of internal controls for the Luminex instrument as part of a multiplexed seven-analyte viral respiratory antibody profile” Clinical and Diagnostic Laboratory Immunology 9:41-45; Kellar and Iannone (2002) “Multiplexed microsphere-based flow cytometric assays” Experimental Hematology 30:1227-1237; Oliver et al. (1998) “Multiplexed analysis of human cytokines by use of the FlowMetrix system” Clinical Chemistry 44:2057-2060; Gordon and McDade (1997) “Multiplexed quantification of human IgG, IgA, and IgM with the FlowMetrix™ system” Clinical Chemistry 43:1799-1801; U.S. Pat. No. 5,981,180 entitled “Multiplexed analysis of clinical specimens apparatus and methods” to Chandler et al. (Nov. 9, 1999); U.S. Pat. No. 6,449,562 entitled “Multiplexed analysis of clinical specimens apparatus and methods” to Chandler et al. (Sep. 10, 2002); and references therein.

Methods of analyzing microsphere populations (e.g. methods of identifying microsphere subsets by their size and/or fluorescence characteristics, methods of using size to distinguish microsphere aggregates from single uniformly sized microspheres and eliminate aggregates from the analysis, methods of detecting the presence or absence of a fluorescent label on the microsphere subset, and the like) are also well described in the literature. See, e.g., the above references.

Suitable instruments, software, and the like for analyzing microsphere populations to distinguish subsets of microspheres and to detect the presence or absence of a label (e.g., a fluorescently labeled label probe) on each subset are commercially available. For example, flow cytometers are widely available, e.g., from Becton-Dickinson ((www.) bd.com) and Beckman Coulter ((www.) beckman.com). Luminex 100™ and Luminex HTS™ systems (which use microfluidics to align the microspheres and two lasers to excite the microspheres and the label) are available from Luminex Corporation ((www.) luminexcorp.com); the similar Bio-Plex™ Protein Array System is available from Bio-Rad Laboratories, Inc. ((www.) bio-rad.com). A confocal microplate reader suitable for microsphere analysis, the FMAT™ System 8100, is available from Applied Biosystems ((www.) appliedbiosystems.com).

As another example of particles that can be adapted for use in the present invention, sets of microbeads that include optical barcodes are available from CyVera Corporation ((www.) cyvera.com). The optical barcodes are holographically inscribed digital codes that diffract a laser beam incident on the particles, producing an optical signature unique for each set of microbeads.

### Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

### Polynucleotide Synthesis

Methods of making nucleic acids (e.g., by in vitro amplification, purification from cells, or chemical synthesis), methods for manipulating nucleic acids (e.g., by restriction enzyme digestion, ligation, etc.) and various vectors, cell lines and the like useful in manipulating and making nucleic acids are described in the above references. In addition, methods of making branched polynucleotides (e.g., amplification multimers) are described in U.S. Pat. No. 5,635,352, U.S. Pat. No. 5,124,246, U.S. Pat. No. 5,710,264, and U.S. Pat. No. 5,849,481, as well as in other references mentioned above.

In addition, essentially any polynucleotide (including, e.g., labeled or biotinylated polynucleotides) can be custom or standard ordered from any of a variety of commercial sources, such as The Midland Certified Reagent Company ((www.) mcrc.com), The Great American Gene Company ((www.) genco.com), ExpressGen Inc. ((www.) expressgen.com), Qiagen (oligos.qiagen.com) and many others.

A label, biotin, or other moiety can optionally be introduced to a polynucleotide, either during or after synthesis. For example, a biotin phosphoramidite can be incorporated during chemical synthesis of a polynucleotide. Alternatively, any nucleic acid can be biotinylated using techniques known in the art; suitable reagents are commercially available, e.g., from Pierce Biotechnology ((www.) piercenet.com). Similarly, any nucleic acid can be fluorescently labeled, for example, by using commercially available kits such as those from Molecular Probes, Inc. ((www.) molecularprobes.com) or Pierce Biotechnology ((www.) piercenet.com) or by incorporating a fluorescently labeled phosphoramidite during chemical synthesis of a polynucleotide.

### Arrays

In an array of capture probes on a solid support (e.g., a membrane, a glass or plastic slide, a silicon or quartz chip, a plate, or other spatially addressable solid support), each capture probe is typically bound (e.g., electrostatically or covalently bound, directly or via a linker) to the support at a unique selected location. Methods of making, using, and analyzing such arrays (e.g., microarrays) are well known in the art. See, e.g., Baldi et al. (2002) DNA Microarrays and Gene Expression: From Experiments to Data Analysis and Modeling, Cambridge University Press; Beaucage (2001) “Strategies in the preparation of DNA oligonucleotide arrays for diagnostic applications” Curr Med Chem 8:1213-1244; Schena, ed. (2000) Microarray Biochip Technology, pp. 19-38, Eaton Publishing; technical note “Agilent SurePrint Technology: Content centered microarray design enabling speed and flexibility” available on the web at chem.agilent.com/temp/rad01539/00039489.pdf; and references therein. Arrays of pre-synthesized polynucleotides can be formed (e.g., printed), for example, using commercially available instruments such as a GMS 417 Arrayer (Affymetrix, Santa Clara, Calif.). Alternatively, the polynucleotides can be synthesized at the selected positions on the solid support; see, e.g., U.S. Pat. No. 6,852,490 and U.S. Pat. No. 6,306,643, each to Gentanlen and Chee entitled “Methods of using an array of pooled probes in genetic analysis.”

Suitable solid supports are commercially readily available. For example, a variety of membranes (e.g., nylon, PVDF, and nitrocellulose membranes) are commercially available, e.g., from Sigma-Aldrich, Inc. ((www.) sigmaaldrich.com). As another example, surface-modified and pre-coated slides with a variety of surface chemistries are commercially available, e.g., from TeleChem International ((www.) arrayit.com), Corning, Inc. (Corning, N.Y.), or Greiner Bio-One, Inc. ((www.) greinerbiooneinc.com). For example, silanated and silyated slides with free amino and aldehyde groups, respectively, are available and permit covalent coupling of molecules (e.g., polynucleotides with free aldehyde, amine, or other reactive groups) to the slides. As another example, slides with surface streptavidin are available and can bind biotinylated capture probes. In addition, services that produce arrays of polynucleotides of the customer's choice are commercially available, e.g., from TeleChem International ((www.) arrayit.com) and Agilent Technologies (Palo Alto, Calif.).

Suitable instruments, software, and the like for analyzing arrays to distinguish selected positions on the solid support and to detect the presence or absence of a label (e.g., a fluorescently labeled label probe) at each position are commercially available. For example, microarray readers are available, e.g., from Agilent Technologies (Palo Alto, Calif.), Affymetrix (Santa Clara, Calif.), and Zeptosens (Switzerland).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of detecting a target nucleic acid sequence, which comprises:
providing a sample comprising or suspected of comprising a target nucleic acid sequence;
incubating at least two label extender probes each comprising a different L-1 sequence, and a label probe system with the sample comprising or suspected of comprising the target nucleic acid sequence; and
detecting whether the label probe system is associated with the sample.

2. The method according to claim 1, wherein the sample is purified chromosomes and the target is double stranded DNA.

3. The method according to claim 1, wherein the sample comprises or is suspected of comprising a target nucleic acid comprising at least two single nucleotide polymorphisms.

4. The method according to claim 1, wherein the label extender probes comprise an allele-specific label probe and a non-allele-specific probe and wherein the non-allele-specific probe is designed to hybridize less stringently to the complimentary strand of the target sequence.

5. The method according to claim 1, wherein the at least one L-1 sequence comprises one or more locked nucleic acids.

6. The method according to claim 5, wherein the one or more locked nucleic acid(s) is/are constrained ethyl nucleic acid(s) (cEt).

7. The method according to claim 1, wherein the target nucleic acid sequence comprises one or more single nucleotide polymorphisms.

8. The method according to claim 1, wherein incubating at least two label extender probes each comprising a different L-1 sequence comprises incubating at least eight label extender probes each comprising a different L-1 sequence, wherein the at least eight label extender probes comprise two sets of four complimentary label extender probes designed to bind double stranded DNA target sequences, and wherein one set of four complimentary label extender probes binds downstream or upstream of the other set of four complimentary label extender probes.

9. The method according to claim 8, wherein each set of four complimentary label extender probes associates with a different set of label probes and each different set of label probes comprise a detectably distinguishable label.

10. A method of detecting a pri-microRNA, which comprises:
providing a sample comprising or suspected of comprising a pri-microRNA;
incubating at least two sets of two label extender probes each comprising a different L-1 sequence, and a label probe system with the sample comprising or suspected of comprising the pri-microRNA, wherein at least one set of L-1 sequences is complementary to a pri-microRNA sequence comprising both stem-loop structure mature sequence and non-stem-loop structure pri-microRNA sequence;
detecting whether the label probe system is associated with the sample.

11. The method according to claim 10, wherein the sample is a tissue.

12. The method according to claim 10, wherein the sample is cells from a cell culture.

13. The method according to claim 10, wherein the cells are human cells.

14. The method according to claim 10, wherein the at least one L-1 sequence comprises one or more locked nucleic acids.

15. The method according to claim 14, wherein the one or more locked nucleic acid(s) is/are constrained ethyl nucleic acid(s) (cEt).

16. The method according to claim 10, wherein the sample comprises both mature miRNA and pri-miRNA and wherein two differently labeled label probe systems are present, thereby detecting whether the sample comprises mature miRNA, immature miRNA or both.

17. The method according to claim 10, wherein at least two different pri-miRNA sequences are in the sample.

18. The method according to claim 10, wherein the label extenders are designed in the cruciform orientation.
